(19) United States
(12) Patent Application Publication
(10) Pub. No.: US 2014/0193824 A1
(43) Pub. Date: Jul. 10, 2014

(54) High-Resolution Clonal Typing of Escherichia coli

(71) Applicants: Evgeni V. SOKURENKO, Seattle, WA (US); James R. JOHNSON, Minneapolis, MN (US); Scott WEISSMAN, Seattle, WA (US); Veronika TCHESNOKOVA, Seattle, WA (US)

(72) Inventors: Evgeni V. SOKURENKO, Seattle, WA (US); James R. JOHNSON, Minneapolis, MN (US); Scott WEISSMAN, Seattle, WA (US); Veronika TCHESNOKOVA, Seattle, WA (US)

(21) Appl. No.: 14/146,427

(22) Filed: Jan. 2, 2014

Related U.S. Application Data
(60) Provisional application No. 61/749,144, filed on Jan. 4, 2013.

(51) Int. Cl. C12Q 1/68

(52) U.S. Cl. CPC C12Q 1/689
USPC 435/6.12; 536/24.33

(57) ABSTRACT

The present invention provides methods and compositions for high-resolution clonal typing of Escherichia coli.

### CROSS REFERENCE

This application is related to U.S. provisional patent application Ser. No. 61/749,144, filed Jan. 4, 2013, the disclosure of which is incorporated by reference herein in its entirety.

### STATEMENT REGARDING FEDERALLY SPONSORED RESEARCH

This invention was made with U.S. government support under K08 award AI057737A, awarded by the National Institutes of Allergy and Infectious Diseases and under ARRA award RC4AI092828, awarded by the National Institutes of Health. The U.S. Government has certain rights in the invention.

### BACKGROUND

Standard multilocus sequence typing (MLST) is usually based on the sequencing of 5 to 8 housekeeping loci in the bacterial chromosome and has provided detailed descriptions of the population structure of bacterial species important to human health. However, even strains with identical MLST profiles (known as sequence types or STs) may possess distinct genotypes, which enable different eco- or pathotypic lifestyles. There is a need for sequence typing that provides a genotyping tool for molecular epidemiology analysis that is more economical than standard 7-locus MLST, but has superior clonal discrimination power and, at the same time, corresponds closely to MLST-based clonal groupings.

### SUMMARY OF THE INVENTION

In a first aspect, the present invention provides a method of typing Escherichia coli in a sample comprising: (a) determining a nucleic acid sequence in the sample of Escherichia coli (E. coli) type 1 fimbrial adhesion (fimH) gene and a further E. coli gene selected from the group consisting of: fumC, adk, gyrB, icd, mdh, purA, and recA to identify a clonotype of the sample; and (b) typing E. coli present in the sample based on the clonotype.

The inventors have surprisingly discovered that methods of the present invention provide the clonal identities of clinical E. coli isolates, which are linked to distinct antimicrobial susceptibility profiles and clinical manifestations. These findings indicate that a clonotype-guided approach substantially reduces the likelihood of drug-bug mismatches during the course of initial antimicrobial therapy by providing more specific data about a patient's actual organism. Furthermore, the methods of the invention provided greater certainty from the outset regarding which antimicrobials can and cannot be used reliably for a given patient with suspected E. coli infection will be of great benefit to patients and health care systems alike.

In other embodiments of the invention, the methods of the invention may be used as a rapid sequence typing scheme for E. coli that preserves the phylogenetic signal, has superior discriminatory power, and resolves clinically important sub-lineages within sequence types. It therefore can serve as a cost-effective alternative to the two most-commonly used clonal typing methods for E. coli, (i) multilocus sequence typing (MLST) and (ii) pulsed-field gel electrophoresis (PFGE), which are poorly suited for associating genetic lineages with susceptibility profiles in clinical practice due to their high costs, slow turnaround times, and/or unsuitably low (for MLST) or variable (for PFGE) levels of discrimination. The clonotyping of the invention is applicable as a molecular tool for both applied and basic investigations regarding the epidemiology and population structure of E. coli.

In some embodiments, the sample is a biological sample from a subject, and the typing indicates presence of antibiotic resistant E. coli in the subject, or is used to diagnose or prognose a disease state in the subject. In some embodiments, the typing indicates that the subject is infected with antibiotic resistant E. coli; and/or the subject is at risk of having a urinary tract infection or sepsis, and the typing is used to diagnose and/or prognose a urinary tract infection or sepsis in the subject. In additional embodiments, the typing indicates efficacy of an antibiotic treatment (e.g., ampicillin (AMP), tetracycline (TET), ampicillin-sulbactam (A/S), trimethoprimsulfamethoxazole (T/S), amoxicillin-clavulanate (A/K), cefazolin (CZ), ciprofloxacin (CIP), gentamicin (GM), nitrofurantoin (NIT), ceftriaxone (CTR) and/or piperacillin-tazobactam (PTZ)) for the subject.

In one embodiment of the invention, the typing is carried out after the subject has undergone treatment for the disease state or the infection with antibiotic resistant E. coli, and the typing indicates efficacy of the treatment. In other embodiments of the invention, the biological sample, for example, is urine, blood, wound, tissue, saliva, sputum, feces, spinal fluid, plasma, peritoneal fluid, ascites, pleural fluid, joint fluid, abscess material, pus, tracheal secretions, bile, exudate, corneal scraping, bone, drainage and biopsy material. In one embodiment, the biological sample is urine.

In one embodiment, the portion of the fimH gene is amplified by an oligonucleotide primer pair consisting of 5′-CACTCAGGGAACCATTCAGGCA-3′ (SEQ ID NO: 01) and 5′-CTTATTGATAAACAAAAGTCAC-3′ (SEQ ID NO: 02). In another embodiment, the portion of the fumC gene is amplified by an oligonucleotide primer pair that amplifies an 500 nucleotide fragment or less of fumC.

In a second aspect, the invention provides a composition consisting of between 2-5 oligonucleotide primer pairs, wherein: (a) a first primer pair selectively amplifies a region of a fimH gene; and (b) a second primer pair selectively amplifies a region of a gene selected from the group consisting of fumC, adk, gyrB, icd, mdh, purA, and recA. In some embodiments of the second aspect of the invention, the first primer pair consists of SEQ ID NO: 01 and SEQ ID NO: 02.

### BRIEF DESCRIPTION OF THE DRAWINGS

The disclosed exemplary aspects have other advantages and features which will be more readily apparent from the detailed description, the appended claims, and the accompanying figures. A brief description of the figures is below.

FIG. 1 shows a sequence-based typing of a collection of 191 model E. coli isolates. (Left panel) Dendrogram of concatenated 7-locus MLST sequences. (Right panel) Dendrogram of full-length type 1 fimbrial adhesin gene fimH sequences, with fimH typing region (fimHTR) alleles and amino acid polymorphisms differed from the consensus structure. Cross-connecting lines link same-strain MLST and fimH haplotypes. The scales at the bottoms of dendrograms indicate phylogenetic distance expressed as percent identity at the nucleotide level. ST numbering is according to the MLST database (on the World Wide Web mlst.uccle/mlst/dbs/Ecoli). The number of isolates associated with the specified type (in parentheses) is shown only where that number is >1. Empty circles indicate STs that include a fimH-null strain. Colors of cross-connecting lines between dendrograms correspond to the colors of the phylogenetic group origins: red, group B2 only; blue, group B1 only; orange, group A only; green, group D only; black, two or more phylogenetic groups. FimH hot spot polymorphisms are underlined. Mature FimH peptide polymorphisms encoded outside the typing region are italicized.

FIG. 2 shows a sliding-window nucleotide polymorphism plot of 7 MLST loci, as well as the fimH lectin and pilin domains. The signal peptide and two fimH domains (lectin and pilin) are partitioned by a vertical dashed line. The red bold fimH typing region (fimHTR) includes the entire fimH lectin domain and small portions of the adjacent regions (i.e., signal peptide to the left, pilin domain to the right). Overlapping windows of 100 nt with a step size of 50 nt were used. The average n value(±the standard error) is shown for each locus

FIG. 3 shows a distribution of isolates and unique profiles by group size among 853 current E. coli strains. ST (FIG. 3A) or CHT (FIG. 3B) sizes: <0.5%, small; 0.5 to 5%, medium; >5%, large. Light bars, total number of strains in each category (left axis). Dark bars, total number of unique profiles in each category (right axis). The axis scale is the same in both panels.

FIG. 4 shows correspondence of fumC-fimH profiles (CHTs) with STs for the 5 largest ST complexes. Dotted lines connect minor STs with the corresponding CHTs; the remaining CHTs correspond to the predominant ST within the complex. CHT circles without a pie slice represent profiles with a total match to the ST complex; circles with a pie slice represent CHTs that mostly (93 to 97%) match the ST complex (the slice symbolizes the “nonmatch” isolates).

FIG. 5 shows the distribution of isolates by clonotype at different laboratories. The outer ring shows the distribution of clonotypes among isolates from all locations combined (Total), in the order of overall clonotype prevalence. The inner rings show the distribution of clonotypes within individual laboratories, sorted within each ring according to local clonotype prevalence. Clonotypes accounting for >1% of the total collection are shade coded consistently across sites.

FIG. 6 shows the antimicrobial resistance profiles within the total collection. Cumulative resistance profiles of individual major CH clonotypes (those with greater than or equal to 1% of isolates) are shown, as well as all other clonotypes combined (37% of isolates), and the total population (Total). The size (number of isolates) of each major clonotype is shown at the lower right. Resistance prevalence value significantly higher (OR greater than or equal to 2) or lower (OR less than or equal to 0.5) than the mean for the rest of the population at the P<0.05 level are marked in dark gray or asterisk (*), respectively.

FIG. 7 shows the association of recurrence or sepsis with major CH clonotypes. Clonotypes are shown in order of overall resistance prevalence, as in FIG. 2. The drug-bug mismatch bar demonstrates the association of recurrence or sepsis with resistance to the prescribed antimicrobial. Significantly increased (*) or decreased (**) recurrence and/or sepsis in a particular CH clonotype with a P value of <0.05; significantly increased (+) or decreased (++) recurrence and/or sepsis in a particular CH clonotype with a P value of <0.10.

FIG. 8 shows CH clonotyping of E. coli in patients' urine samples. (A) Detection of CH40-30 clones in urine samples using quantitative polymerase chain reaction (qPCR) with RMTS1 gene-specific probe. (B) Detection of CH40-30 clones in urine samples using qPCR with fimH SNP-specific probe. Determination of three different CH clonotypes ((C) CH40-30; (D) CH35-27; and (E) CH24-10) in urine samples using pyrosequencing of fumC and fimH regions on PyroMark Q24.

FIG. 9 shows across laboratory comparisons of cumulative antimicrobial profiles for 10 major CH clonotypes. Clonotypes with increased (OR greater than or equal to 2.0) or decreased (OR less than or equal to 0.5) resistance prevalence relative to the rest of the population are shown in dark gray and asterisk (*), respectively (at the P<0.10 level, due to the relatively low number of isolates). Y axis shows the prevalence of resistance to each antimicrobial. Ch=Children's Hospital (Seattle); GH=Group Health Cooperative (Seattle); UW=University of Washington Medical Center (Seattle); HV=Harborview Medical Center (Seattle); and VA=VA Medical Center (Minneapolis).

### DETAILED DESCRIPTION OF THE INVENTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M.P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

Terms used in the claims and specification are defined as set forth below unless otherwise specified. In the case of direct conflict with a term used in a parent provisional patent application, the term used in the instant specification shall control.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of skill in the art, such as the Oxford Dictionary of Biochemistry and Molecular Biology (Ed. Anthony Smith, Oxford University Press, Oxford, 2004).

As used herein, the singular forms “a”, “an” and “the” include plural referents unless the context clearly dictates otherwise. “And” as used herein is interchangeably used with “or” unless expressly stated otherwise.

All embodiments disclosed herein can be used in combination unless the context clearly dictates otherwise.

In a first aspect, the present invention provides a method of typing Escherichia coli in a sample comprising: (a) determining a nucleic acid sequence in the sample of Escherichia coli (E. coli) type 1 fimbrial adhesion (fimH) gene and a further E. coli gene selected from the group consisting of: fumC, adk, gyrB, icd, mdh, purA, and recA to identify a clonotype of the sample; and (b) typing E. coli present in the sample based on the clonotype.

The inventors have surprisingly discovered that methods of the present invention provide the clonal identities of clinical E. coli isolates, which are linked to distinct antimicrobial susceptibility profiles and clinical manifestations. These findings indicate that a clonotype-guided approach substantially reduces the likelihood of treating an E. coli infection with an ineffective antimicrobial agent (a drug-bug mismatch) during the course of initial antimicrobial therapy by providing more specific data about a patient's actual infectious organism. Furthermore, the methods of the invention provide greater certainty regarding which antimicrobials can and cannot be used reliably for a given subject with suspected E. coli infection, and will thus be of great benefit to patients and health care systems alike.

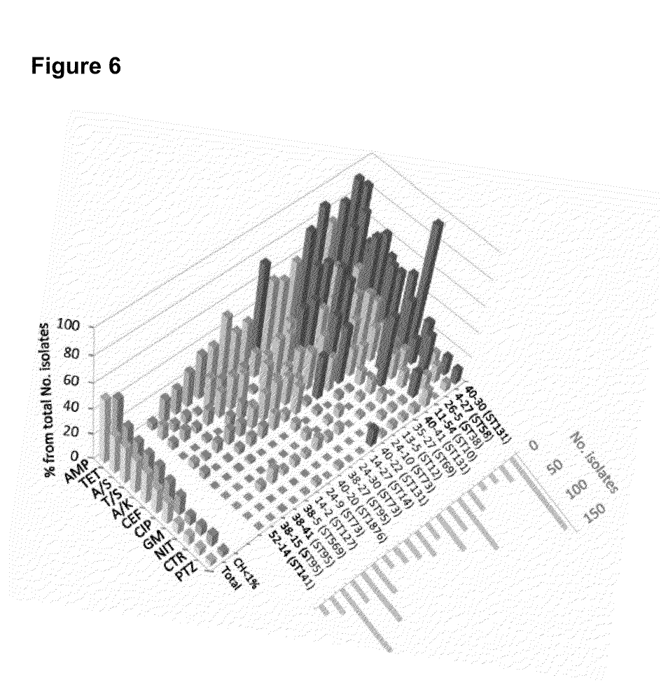

In other embodiments of the invention, the methods of the invention may be used as a rapid sequence typing scheme for E. coli that preserves the phylogenetic signal (defined herein as the statistical non-independence among species trait values due to their phylogenetic relatedness), has superior discriminatory power compared to standard 7-locus MLST, and resolves clinically important sub-lineages within sequence types (for example, the clonotype designated CH40-30 is a sublineage of sequence type ST131; see FIG. 6 and the examples that follow). It therefore can serve as a cost-effective alternative to the two most-commonly used clonal typing methods for E. coli, (i) standard multilocus sequence typing (MLST) of 5-8 housekeeping loci and (ii) pulsed-field gel electrophoresis (PFGE), which are poorly suited for associating genetic lineages with susceptibility profiles in clinical practice due to their high costs, slow turnaround times, and/or unsuitably low (for MLST) or variable (for PFGE) levels of discrimination. The clonotyping of the invention is applicable as a molecular tool for both applied and basic investigations regarding the epidemiology and population structure of E. coli.

The type 1 fimbrial adhesin (fimH) gene is involved in regulation of length and mediation of adhesion of type 1 fimbriae (but not necessary for the production of fimbriae). An exemplary sequence for fimH, includes, but is not limited to that of Escherichia coli strain ECOR63 type 1 fimbrial adhesin (fimH) gene, corresponding to GenBank: FJ865645.1: gi|268638760|gb|FJ865645.1|Escherichia coli strain ECOR63 type 1 fimbrial adhesin (fimH) gene (SEQ ID NO: 04):

ATGAAACGAGTTATTACCCTGTTTGCTGTACTGCTGATGGGCTGGTCGG
TAAATGCCTGGTCATTCGCCTGTAAAACCGCCAATGGTACTGCTATCCC
TATTGGCGGTGGCAGCGCCAATGTTTATGTAAACCTTGCGCCTGCCGTG
AATGTGGGGCAAAACCTGGTCGTGGATCTTTCGACGCAAATCTTTTGCC
ATAACGATTACCCGGAAACCATTACAGACTATGTCACACTGCAACGAGG
TTCGGCTTATGGCGGCGTGTTATCTAGTTTTTCCGGGACCGTAAAATAT
AATGGCAGTAGCTATCCTTTCCCTACTACCAGCGAAACGCCGCGGGTTG
TTTATAATTCGAGAACGGATAAGCCGTGGCCGGTGGCGCTTTATTTGAC
GCCTGTGAGCAGTGCTGGCGGGGTGGCGATTAAAGCTGGTTCATTAATT
GCCGTGCTTATTTTGCGACAGACCAACAACTATAACAGCGATGATTTTC
AGTTTGTGTGGAATATTTACGCCAATAATGATGTGGTGGTGCCCACTGG
CGGCTGTGATGTTTCTGCTCGTGATGTCACCGTTACTCTGCCGGACTAC
CCTGGTTCAGTGCCGATTCCTCTTACCGTTTATTGTGCGAAAAGCCAAA
ACCTGGGGTATTACCTCTCCGGCACAACCGCAGATGCGGGCAACTCGAT
TTTCACCAATACCGCGTCGTTTTCACCCGCGCAGGGCGTCGGCGTACAG
TTGACGCGCAACGGTACGATTATTCCAGCGAATAACACGGTATCGTTAG
GAGCAGTAGGGACTTCGGCGGTAAGTCTGGGATTAACGGCAAATTACGC
ACGTACCGGAGGGCAGGTGACTGCAGGGAATGTGCAATCGATTATTGGC
GTGACTTTTGTTTATCAA.

The fumarase C (fumC) gene is involved in catalysis of the reversible addition of water to fumarate to give L-malate. An exemplary sequence for fumC, includes, but is not limited to that of Escherichia coli ECOR70 fumarase C (fumC) gene, GenBank: AY464329.1; gi|39754439|gb|AY464329.1|Escherichia coli strain ECOR70 fumarase C (fumC) gene, SEQ ID NO: 05:

CGAGCGCCATTCGGCAGGCGGCGGATGAAGTACTGGCAGGACAGCATG
ACGACGAATTCCCGCTGGCTATCTGGCAGACCGGCTCCGGCACGCAAA
GTAACATGAACATGAACGAAGTGCTGGCTAACCGGGCCAGTGAATTAC
TCGGCGGCGTGCGCGGGATGGAACGTAAAGTTCACCCTAACGACGACG
TGAACAAAAGCCAAAGTTCCAACGATGTCTTTCCGACGGCGATGCACG
TTGCGGCGCTGCTGGCGCTGCGCAAGCAACTCATTCCGCAGCTTAAAA
CCCTGACACAGACACTGAGTGAAAAATCGCGTGCATTTGCCGATATCG
TCAAAATCGGTCGAACCCACTTGCAGGACGCCACGCCGCTAACACTAG
GGCAGGAGATTTCCGGCTGGGTAGCGATGCTCGAGCATAATCTCAAAC
ATATCGAATACAGCCTGCCTCACGTAGCGGAACTGGC.

The adenylate kinase (adk) gene is involved in catalysis of the reversible transfer of the terminal phosphate group between ATP and AMP. Adenylate kinase also plays an important role in cellular energy homeostasis and in adenine nucleotide metabolism. An exemplary sequence for adk, includes, but is not limited to that of Escherichia coli strain ECOR70 adenylate kinase (adk) gene, GenBank: AY464327.1; gi|39754301|gb|AY464327.1|Escherichia coli strain ECOR70 adenylate kinase (adk) gene (SEQ ID NO: 06):

GGGGAAAGGGACTCAGGCTCAGTTCATCATGGAGAAATATGGTATTCCG
CAAATCTCCACTGGCGATATGCTGCGTGCTGCGGTCAAATCTGGCTCCG
AGCTGGGTAAACAAGCAAAAGACATTATGGATGCTGGCAAACTGGTCAC
CGACGAACTGGTGATCGCGCTGGTTAAAGAGCGCATTGCTCAGGAAGAC
TGCCGTAATGGTTTCCTGTTGGACGGCTTCCCGCGTACCATTCCGCAGG
CAGACGCGATGAAAGAAGCGGGCATCAATGTTGATTACGTTCTGGAATT
CGACGTACCGGACGAACTGATTGTTGATCGTATCGTAGGCCGCCGCGTT
CATGCGCCGTCTGGTCGTGTTTATCACGTTAAATTCAATCCGCCGAAAG
TAGAAGGCAAAGACGACGTTACCGGTGAAGAACTGACTACCCGTAAAGA
CGATCAGGAAGAAACCGTGCGTAAACGTCTGGTTGAATACCATCAGATG
ACTGCACCGCTGATCGGCTACTACTCCAAAGAAGCGGAAGCGGGTA.

The DNA gyrase subunit B (gyrB) gene belongs to the type II topoisomerase family. An exemplary sequence for gyrB, includes, but is not limited to that of Escherichia coli strain UPEC_{—}156 DNA gyrase B (gyrB) gene, GenBank: JF893048.1; gi|336044839|gb|JF893048.1|Escherichia coli strain UPEC_{—}156 DNA gyrase B (gyrB) gene (SEQ ID NO: 07):

ATGACCGTTCTGCACGCAGGCGGTAAATTTGACGATAACTCCTATAAA
GTGTCCGGCGGTCTGCACGGCGTTGGTGTTTCGGTAGTAAACGCCCTG
TCGCAAAAACTGGAGCTGGTTATCCAGCGCGAGGGTAAAATTCACCGT
CAGATCTACGAACACGGTGTACCGCAGGCTCCGCTGGCGGTTACCGGC
GAGACTGAAAAAACCGGCACCATGGTGCGTTTCTGGCCCAGCCTCGAA
ACCTTCACCAATGTGACCGAGTTCGAATATGAAATTCTGGCGAAACGT
CTGCGTGAGTTGTCGTTCCTCAACTCCGGCGTTTCCATTCGTTTGCGC
GACAAGCGTGACGGCAAAGAAGACCACTTCCACTATGAAGGCGGCATC
AAGGCGTTCGTTGAATATCTGAACAAGAACAAAACGCCGATCCATCCG
AATATCTTCTACTTCTCCACCGAAAAAGACGGTATTGGCGTCGAAGTG
GCGTTGCAGTGGAACGATGGCTTCCAGGAAAACATCTACTGC.

The isocitrate dehydrogenase (icd) gene is an enzyme that catalyzes the oxidative decarboxylation of isocitrate, producing alpha-ketoglutarate (α-ketoglutarate) and CO₂. An exemplary sequence for icd, includes, but is not limited to that of Escherichia coli strain ECOR27 isocitrate dehydrogenase (icd) gene, GenBank: AY132834.1; gi|33383656|gb|AY132834.1|Escherichia coli strain ECOR27 isocitrate dehydrogenase (icd) gene (SEQ ID NO: 08):

ACCCTGCAAAACGGCAAACTCAACGTTCCTGAAAATCCGATTATCCCT
TACATTGAAGGTGATGGAATCGGTGTAGATGTAACCCCAGCCATGCTG
AAAGTGGTCGACGCTGCAGTCGAGAAAGCCTATAAAGGCGAGCGTAAA
ATCTCCTGGATGGAAATTTACACCGGTGAAAAATCCACACAGGTTTAT
GGTCAGGATGTCTGGCTGCCTGCTGAAACCCTTGATCTGATTCGTGAA
TATCGCGTTGCCATTAAAGGTCCGCTGACCACTCCTGTTGGTGGCGGT
ATTCGCTCTCTGAACGTTGCCCTGCGCCAGGAACTGGATCTCTACATC
TGCCTGCGTCCGGTACGTTACTATCAGGGCACTCCAAGCCCGGTTAAA
CACCCTGAACTGACCGATATGGTTATCTTCCGTGAAAACTCGGAAGAC
ATTTATGCGGGTATCGAATGGAAAGCAGACTCTGCCGACGCCGAGAAA
GTGATTAAATTCCTGCGTGAAGAGATGGGGGTGAAGAAAATTCGCTTC
CCGGAACATTGTGGTATCGGTATTAAGCCGTGTTCGGAAGAAGGCACC
AAACGTCTGGTTCGTGCAGCGATCGAATACGCAATTGCTAACGATCGT
GACTCTGTGACTCTGGTGCACAAAGGCAACATCATGAAGTTCACCGAA
GGCGCGTTTAAAGACTGGGGCTACCAGCTGGCGCGTGAAGAGTTTGGC
GGTGAACTGATCGACGGCGGCCCGTGGCTGAAAGTTAAAAACCCGAAC
ACCGGCAAAGAGATCGTCATTAAAGACGTGATTGCTGATGCATTCCTG
CAACAAATCCTGCTGCGTCCGGCTGAATATGATGTTATCGCCTGTATG
AACCTGAACGGTGACTACATTTCTGACGCTCTGGCAGCGCAGGTTGGC
GGTATCGGTATCGCCCCTGGAGCAAACATCGGTGACGAATGCGCCCTG
TTTGAAGCCACCCCCGGTACTGCGCCGAAATACGCCGGTCAGGACAAA
GTAAACCCTGGCTCTATTATTCTCTCCGCTGAGATGATGTTACGCCAT
ATGGGTTGGACTGAAGCGGCTGACCTGATTGTTAAAGGTATGGAAGGC
GCAATCAATGCCAAGACCGTAACCTATGACTTCGAACGTCTGATGGAA
GGCGCTAAACTGCT.

The malate dehydrogenase (mdh) gene is an enzyme that reversibly catalyzes the oxidation of malate to oxaloacetate using the reduction of NAD+ to NADH. An exemplary sequence for mdh, includes, but is not limited to that of Escherichia coli strain DFS179 NAD(P)-binding malate dehydrogenase (mdh) gene, GenBank: HM221406.1; gi|297497101|gb|HM221406.1|Escherichia coli strain DFS179 NAD(P)-binding malate dehydrogenase (mdh) gene (SEQ ID NO: 09):

GGTGAAGATGCGACTCCGGCGCTGGAAGGCGCAGATGTCGTTCTTATC
TCTGCAGGTGTAGCGCGTAAACCGGGTATGGATCGTTCCGACCTGTTT
AACGTTAACGCCGGCATCGTGAAAAACCTGGTACAGCAAGTTGCGAAA
ACCTGCCCGAAAGCGTGCATTGGTATTATCACTAACCCGGTTAACACC
ACAGTTGCAATTGCTGCTGAAGTGCTGAAAAAAGCCGGTGTTTATGAC
AAAAACAAACTGTTCGGCGTTACCACGCTGGATATCATTCGTTCCAAC
ACCTTTGTTGCGGAACTGAAAGGCAAACAGCCAGGCGAAGTTGAAGTG
CCGGTTATTGGCGGTCACTCTGGTGTTACCATTCTGCCGCTGCTGTCA
CAGGTTCCTGGCGTTAGTTTTACCGAGCAGGAAGTGGCTGATCTGACC
AAACGTATCCAGAACGCGGGTACTGAGGTGGTTGAAGCGAAAGCCGGT
GGCGGGTCTGCAACCCTGTCTATGGGCCAGGCAGCTGCACGTTTTGGT
CTGTCTCTGGTTCGTGCACTG.

The adenylosuccinate synthetase (purA) gene is an enzyme that plays an important role in purine biosynthesis, by catalysing the guanosine triphosphate (GTP)-dependent conversion of inosine monophosphate (IMP) and aspartic acid to guanosine diphosphate (GDP), phosphate and N(6)-(1,2-dicarboxyethyl)-AMP. An exemplary sequence for purA, includes, but is not limited to that of Escherichia coli strain APEC_{—}173 adenylosuccinate synthetase (purA) gene, GenBank: JF892777.1; gi|335328288|gb|JF892777.1|Escherichia coli strain APEC_{—}173 adenylosuccinate synthetase (purA) gene (SEQ ID NO: 10):

TCCGAAGCATGTCCGCTGATCCTTGATTATCACGTTGCGCTGGATAACGC
GCGTGAGAAAGCGCGTGGCGCGAAAGCGATCGGCACCACCGGTCGTGGTA
TCGGGCCTGCTTATGAAGATAAAGTGGCACGTCGCGGTCTGCGTGTTGGC
GACCTTTTCGACAAAGAAACCTTCGCTGAAAAACTGAAAGAAGTGATGGA
ATATCACAACTTCCAGTTGGTTAACTACTACAAAGCTGAAGCGGTTGATT
ACCAGAAAGTTCTGGATGATACGATGGCTGTTGCCGACATCCTGACTTCT
ATGGTGGTTGACGTTTCTGACCTGCTCGACCAGGCGCGTCAGCGTGGCGA
TTTCGTCATGTTTGAAGGTGCGCAGGGTACGCTGCTGGATATCGACCACG
GTACTTATCCGTACGTAACTTCTTCCAACACCACTGCTGGTGGCGTGGCG
ACCGGTTCCGGCCTGGGCCCGCGTTATGTTGATTACGTTCTGGGTATCCT
CAAAGCTTACTCCACTCGTGTGGGTGCAGGTCCGTTCCCGACTGAACTGT
TTGATGAAACTGGCGAGTTCCTCTGCAAGCAGGGTAACGAATTCGGCGCA
ACTACGGGTCGTCGTCGTCGTACCGGCTGGCTGGACAC.

The RecA protein (recA) gene is a 38 kilodalton protein essential for the repair and maintenance of DNA. An exemplary sequence for recA, includes, but is not limited to that of Escherichia coli strain ECOR70 RecA protein (recA) gene, GenBank: AY464332.1; gi|39754649|gb|AY464332.1|Escherichia coli strain ECOR70RecA protein (recA) gene (SEQ ID NO: 11):

CGCACGTAAACTGGGCGTCGATATCGATAACCTGCTGTGCTCCCAGCCG
GACACCGGCGAGCAGGCACTGGAAATCTGTGACGCCCTGGCGCGTTCTG
GCGCAGTAGACGTTATCGTCGTTGACTCCGTGGCGGCACTGACGCCGAA
AGCGGAAATCGAAGGCGAAATCGGCGACTCTCACATGGGCCTTGCGGCA
CGTATGATGAGCCAGGCGATGCGTAAGCTGGCGGGTAACCTGAAGCAGT
CCAACACGCTGCTGATCTTCATCAACCAGATCCGTATGAAAATTGGTGT
GATGTTCGGTAACCCGGAAACCACTACCGGTGGTAACGCGCTGAAATTC
TACGCCTCTGTTCGTCTCGACATCCGTCGTATCGGCGCGGTGAAAGAGG
GCGAAAACGTGGTGGGTAGCGAAACCCGCGTGAAAGTGGTGAAGAACAA
AATCGCTGCACCGTTTAAACAGGCTGAATTTCAGATCCTCTACGGCGAA
GGTATCAACTTCTACGGCGA.

The vast majority of E. coli strains encode type 1 fimbriae. The fim cluster is located in a highly recombinogenic region on the E. coli chromosome, just downstream of the leuX tRNA locus, into which pathogenicity islands are frequently inserted. The fimH gene, encoding the type 1 fimbrial adhesin, is under positive selection for functional mutations, whereby single nucleotide polymorphisms (SNPs) can produce amino acid replacements that dramatically alter bacterial cell adhesion properties relevant to pathogenesis.

As used herein, the term “clonotype” refers to a clonal group or subspecies of genetically related E. coli lineages based on the nucleotide sequences of the fimH loci and at least one other E. coli gene loci. The inventors have discovered the use of a clonotype (i.e. clonotyping) based on the fimH sequence and at least one other E. coli gene loci as a predictive marker for antimicrobial susceptibility, and that the clonotyping methods of the invention are superior to the two most-commonly used sequence typing methods for E. coli, standard multilocus sequence typing of 5-8 housekeeping loci (MLST) and pulsed-field gel electrophoresis (PFGE), which are poorly suited for associating genetic lineages with susceptibility profiles in clinical practice due to their high costs, slow turnaround times, and/or unsuitably low (for MLST) or variable (for PFGE) levels of discrimination.

Any suitable sample can be used in which E. coli may be present and from which it would be useful to identify the subspecies of E. coli present. In various non-limiting embodiments, the sample may be an environmental sample (e.g., water, seawater, soil, food or food item, agricultural, surface swab, medical supplies or devices), a clinical sample or a biological sample (e.g., blood, plasma, serum, lymph node, gastrointestinal tissue, urine, exudates, other body fluids, or any other plant or animal tissue), any microbial culture or any microbial colony. In one embodiment of the invention, the sample is a biological sample. Biological examples include, but are not limited to urine, blood, wound, tissue, saliva, sputum, feces, spinal fluid, plasma, peritoneal fluid, ascites, pleural fluid, joint fluid, abscess material, pus, tracheal secretions, bile, exudate, corneal scraping, bone, drainage, lymph fluid, and biopsy material. In one embodiment, the biological sample is urine.

In some embodiments, the sample is a biological sample from a subject, and the typing indicates presence of antibiotic resistant E. coli in the subject, or is used to diagnose or prognose a disease state in the subject. In one embodiment, the methods of the invention comprise diagnosing whether a sample contains an antibiotic resistant strain of E. coli. As used herein, “antibiotic resistant” or “antibiotic resistance” indicates a sub-population or subspecies or clonal group of E. coli, that are able to survive after exposure to one or more antibiotics, antibiotic treatments or therapies or antimicrobials. Antibiotic resistance is a serious and growing phenomenon in contemporary medicine and has emerged as one of the pre-eminent public health concerns of the 21st century.

The term “subject” or “patient” as used herein includes both humans and non-humans and include, but is not limited to humans, non-human primates, canines, felines, murines, bovines, equines, and porcines.

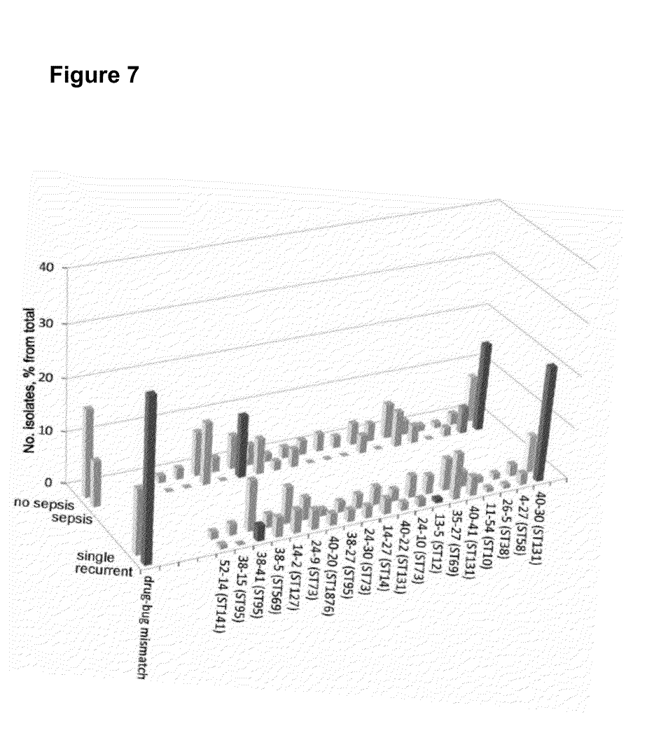

In some embodiments, the typing indicates that the subject is infected with antibiotic resistant E. coli; and/or the subject has or is at risk of having a urinary tract infection or sepsis, and the typing is used to diagnose and/or prognose a urinary tract infection or sepsis in the subject. For example, the CH40-30, CH4-27, CH11-54, CH13-5, CH40-22 and CH35-27 clonotypes are extensively resistant to certain antimicrobials (e.g. resistant to AMP, TET, A/S, T/S, A/K, CEF or CIP), while the CH38-41, CH38-15, CH52-14, CH24-9 and CH14-2 clonotypes are extensively susceptible to certain antimicrobials (e.g. sensitive to AMP, TET, A/S, T/S, A/K, CEF or CIP) (also see FIG. 6). Additionally, the CH40-30, CH14-2 and CH4-27 clonotypes were significantly overrepresented among patients with persistent or recurrent UTIs or sepsis (see FIG. 7), while the CH38-41, CH13-5, CH38-15 and CH24-10 clonotypes were less likely to associate with UTI persistence or recurrence of infection. For example, for a UTI patient identified to be infected with the CH40-30 clonotype the prognosis would be likely that the patient will have recurrence of the UTI. In another example, a patient who is identified to be infected with the CH14-2 clonotype the prognosis would be likely that the patient develop sepsis.

In another embodiment, diagnosing a UTI may also comprise identifying the clonotype (e.g. CH40-30, CH4-27, CH11-54, CH13-5, CH40-22, CH35-27, CH38-41, CH38-15, CH52-14, CH24-9, CH14-2 or any of the clonotypes identified in Table A below) from the biological sample of the subject. Additional methods may comprise detecting one or more clonotypes in a patient being treated for UTI, where you can compare the presence of one or more clonal subspecies from a patient or subject being treated for UTI to a control (for example, a control may be the presence of the clonal subspecies in the patient or subject at baseline before treatment or the absence of the clonal subspecies in a patient or subject not suspected of having a UTI).

In another embodiment, diagnosing sepsis may also comprise identifying the clonotype (e.g. CH40-30, CH4-27, CH11-54, CH13-5, CH40-22, CH35-27, CH38-41, CH38-15, CH52-14, CH24-9, CH14-2 or any of the clonotypes identified in Table A below) from the biological sample of the subject. Additional methods may comprise detecting one or more clonotypes in a patient being treated for sepsis, where you can compare the presence of one or more clonal subspecies from a patient or subject being treated for sepsis to a control (for example, a control may be the presence of the clonal subspecies in the patient or subject at baseline before treatment or the absence of the clonal subspecies in a patient or subject not suspected of having sepsis).

In a further embodiment, the methods of the invention may be used for treating a patient or subject, wherein the methods may further comprise carrying out a clinical step based on the clonotype. In such an example, if the identified clonotype is predicted to have susceptibility to a specific antibiotic treatment, then methods may further comprise administering the antibiotic treatment to the patient or subject. However, if the identified clonotype is predicted to have resistance to a specific antibiotic treatment, then methods may further comprise rejecting administration of the antibiotic treatment to the patient or subject. In a non-limiting example, if the CH40-30 clonotype is identified in a biological sample from a patient, then treating that patient with ampicillin would be rejected by a clinician, while treating that patient with nitrofurantoin would be allowed. In another non-limiting example, if the CH38-15 clonotype is identified in a biological sample from a patient, then treating that patient with ampicillin would be allowed by a clinician. In such methods of treating a patient, the patient may be: (i) suspected of having, (ii) previously diagnosed with, or (iii) currently being treated for; a UTI, sepsis or any infection with an antibiotic resistant E. coli.

In a yet further embodiment, the methods of the invention may be used for treating a patient or subject, wherein the method may further comprise:

(a) determining a clonotype from a sample of a subject or patient;

(b) providing an indication that the clonotype is:
(i) resistant to an antibiotic agent when the clonotype indicates that less than 80% of the isolates in that clonotype are susceptible to that antibiotic agent; or
(ii) susceptible to an antibiotic agent when the clonotype indicates that greater than or equal to 80% of the isolates in that clonotype are susceptible to that antibiotic agent; and

(c) rejecting treatment with the antibiotic agent if the clonotype indicates it is resistant to that antibiotic agent, or

(d) allowing treatment with the antibiotic agent if the clonotype indicates is susceptible to that antibiotic agent. In a non-limiting example, if it is determined that a subject or patient is infected with the CH40-30 clonotype, then treating that patient with ampicillin would be rejected by a clinician. In another non-limiting example, if it is determined that a subject or patient is infected with the CH38-15 clonotype, then treating that patient with ampicillin would be allowed by a clinician.

TABLE A
Clonotypes and sequences of 19 major CH clonotypes (also see FIGS. 6-7)

| CH clonotype | fimH and fumC sequences of CH clonotypes |
|---|---|
| CH40-30 | fumC_allele 40 (SEQ ID NO: 12) |
| | CGAGCGCCATTCGGCAGGCGGCGGATGAAGTACTGGCAGGACAGCATGACGACGAA |
| | TTCCCGCTGGCTATCTGGCAGACCGGCTCCGGCACGCAAAGTAACATGAATATGAA |
| | CGAAGTGCTGGCTAACCGGGCCAGTGAATTACTCGGTGGCGTGCGCGGGATGGAAC |
| | GTAAAGTTCACCCTAACGACGACGTGAACAAAAGCCAAAGTTCCAACGATGTCTTT |
| | CCGACGGCGATGCACGTTGCGGCACTACTGGCGCTGCGCAAGCAACTCATTCCACA |
| | ACTTAAAACCCTGACCCAGACGCTGAGTGAAAAATCCCGCGCATTTGCTGATATCG |
| | TCAAAATCGGTCGTACCCACTTGCAGGACGCCACGCCGTTAACGCTGGGGCAGGAG |
| | ATTTCCGGCTGGGTAGCGATGCTGGAGCATAATCTCAAACATATCGAATACAGCCT |
| | GCCTCACGTAGCGGAACTGGC |
| | fimH_allele 30 (SEQ ID NO: 13) |
| | TTCGCCTGTAAAACCGCCAATGGTACCGCTATTCCTATTGGCGGTGGCAGCGCTAA |
| | TGTTTATGTAAACCTTGCGCCTGCCGTGAATGTGGGGCAAAACCTGGTCGTAGATC |
| | TTTCGACGCAAATCTTTTGCCATAACGATTATCCGGAAACCATTACAGACTATGTC |
| | ACACTGCAACGAGGCTCGGCTTATGGCGGCGTGTTATCTAATTTTTCCGGGACCGT |
| | AAAATATAGTGGCAGTAGCTATCCATTTCCGACTACCAGCGAAACGCCGCGGGTTG |
| | TTTATAATTCGAGAACGGATAAGCCGTGGCCGGTGGCGCTTTATTTGACGCCTGTG |
| | AGCAGTGCGGGTGGGGTGGCGATTAAAGCTGGCTCATTAATTGCCGTGCTTATTTT |
| | GCGACAGACCAACAACTATAACAGCGATGATTTCCAGTTTGTGTGGAATATTTACG |
| | CCAATAATGATGTGGTGGTGCCTACTGGCGGCTGCGATGTT |
| CH4-27 | fumC_allele 4 (SEQ ID NO: 14) |
| | CGAGCGCCATTCGGCAGGCGGCGGATGAAGTACTGGCAGGACAGCATGACGACGAA |
| | TTCCCGCTGGCTATCTGGCAGACCGGCTCCGGCACGCAAAGTAACATGAACATGAA |
| | CGAAGTGCTGGCTAACCGGGCCAGTGAATTACTCGGCGGCGTGCGCGGGATGGAAC |
| | GTAAAGTTCACCCTAACGACGACGTGAACAAAAGCCAAAGTTCCAACGATGTCTTT |
| | CCGACGGCGATGCACGTTGCGGCGCTGCTGGCGCTGCGCAAGCAACTCATTCCGCA |
| | GCTTAAAACCCTGACACAGACACTGAGTGAAAAATCGCGTGCATTTGCCGATATCG |
| | TCAAAATCGGTCGAACCCACTTGCAGGACGCCACGCCGCTAACACTAGGGCAGGAG |
| | ATTTCCGGCTGGGTAGCGATGCTCGAGCATAATCTCAAACATATCGAATACAGCCT |
| | GCCTCACGTAGCGGAACTGGC |
| | fimH_allele 27 (SEQ ID NO: 15) |
| | TTCGCCTGTAAAACCGCCAATGGTACCGCTATCCCTATTGGCGGTGGCAGCGCCAA |
| | TGTTTATGTAAACCTTGCGCCCGTCGTGAATGTGGGGCAAAACCTGGTCGTGGATC |
| | TTTCGACGCAAATCTTTTGCCATAACGATTATCCGGAAACCATTACAGACTATGTC |
| | ACACTGCAACGAGGCTCGGCTTATGGCGGCGTGTTATCTAATTTTTCCGGGACCGT |
| | AAAATATAGTGGCAGTAGCTATCCATTTCCTACCACCAGCGAAACGCCGCGCGTTG |
| | TTTATAATTCGAGAACGGATAAGCCGTGGCCGGTGGCGCTTTATTTGACGCCTGTG |
| | AGCAGTGCGGGCGGGGTGGCGATTAAAGCTGGCTCATTAATTGCCGTGCTTATTTT |
| | GCGACAGACCAACAACTATAACAGCGATGATTTCCAGTTTGTGTGGAATATTTACG |
| | CCAATAATGATGTGGTGGTGCCTACTGGCGGCTGCGATGTT |
| CH26-5 | fumC_allele 26 (SEQ ID NO: 16) |
| | CGAGCGCCATTCGGCAGGCGGCGGATGAAGTACTGGCAGGACAGCATGACGACGAA |
| | TTCCCATTGGCTATCTGGCAGACCGGCTCCGGCACGCAAAGTAACATGAACATGAA |
| | CGAAGTGCTGGCTAACCGGGCCAGTGAATTACTCGGCGGCGTGCGTGGGATGGAGC |
| | GTAAAGTTCACCCTAACGACGACGTGAACAAAAGCCAAAGTTCTAACGATGTCTTT |
| | CCAACGGCGATGCACGTTGCGGCGCTGCTGGCGCTGCGCAAGCAACTCATTCCGCA |
| | GCTTAAAACCCTGACACAGACGCTGAGTGAAAAATCCCGTGCATTTGCCGATATCG |
| | TAAAAATCGGTCGAACCCACTTGCAGGACGCCACGCCGCTAACACTGGGGCAGGAG |
| | ATTTCCGGCTGGGTAGCGATGCTCGAGCATAATCTCAAACATATTGAATACAGCCT |
| | GCCTCACGTAGCGGAACTGGC |
| | fimH_allele5 (SEQ ID NO: 17) |
| | TTCGCCTGTAAAACCGCCAATGGTACCGCTATCCCTATTGGCGGTGGCAGCGCCAA |
| | TGTTTATGTAAACCTTGCGCCTGCCGTGAATGTGGGGCAAAACCTGGTCGTGGATC |
| | TTTCGACGCAAATCTTTTGCCATAACGATTACCCGGAAACCATTACAGACTATGTC |
| | ACACTGCAACGAGGTTCGGCTTATGGCGGCGTGTTATCTAGTTTTTCCGGGACCGT |
| | AAAATATAATGGCAGTAGCTATCCTTTCCCTACTACCAGCGAAACGCCGCGGGTTG |
| | TTTATAATTCGAGAACGGATAAGCCGTGGCCGGTGGCGCTTTATTTGACGCCTGTG |
| | AGCAGTGCGGGGGGAGTGGCGATTAAAGCAGGCTCATTAATTGCCGTGCTTATTTT |
| | GCGACAGACCAACAACTATAACAGCGATGATTTCCAGTTTGTGTGGAATATTTACG |
| | CCAATAATGATGTGGTGGTGCCCACTGGCGGCTGCGATGTT |
| CH11-54 | fumcC_allele 11 (SEQ ID NO: 18) |
| | CGAGCGCCATTCGTCAGGCGGCGGATGAAGTACTGGCAGGACAGCATGACGACGAA |
| | TTCCCGCTGGCTATCTGGCAGACCGGCTCCGGCACGCAAAGTAACATGAACATGAA |
| | CGAAGTGCTGGCTAACCGGGCCAGTGAATTACTCGGCGGTGTGCGCGGGATGGAAC |
| | GTAAAGTTCACCCTAACGACGACGTGAACAAAAGCCAAAGTTCCAACGATGTCTTT |
| | CCGACGGCGATGCACGTTGCGGCGCTGCTGGCGCTGCGCAAGCAACTCATTCCTCA |
| | GCTTAAAACCCTGACACAGACACTGAATGAGAAATCCCGTGCTTTTGCCGATATCG |
| | TCAAAATTGGTCGTACTCACTTGCAGGATGCCACGCCGTTAACGCTGGGGCAGGAG |
| | ATTTCCGGCTGGGTAGCGATGCTCGAGCATAATCTCAAACATATCGAATACAGCCT |
| | GCCTCACGTAGCGGAACTGGC |
| | fimH_allele 54 (SEQ ID NO: 19) |
| | TTCGCCTGTAAAACCGCCAATGGCACCGCTATCCCTATTGGCGGTGGCAGCGCCAA |
| | TGTTTATGTAAACCTTGCGCCCGCCGTGAATGTGGGGCAAAACCTGGTCGTGGATC |
| | TTTCGACGCAAATCTTTTGCCATAACGATTACCCGGAAACCATTACAGATTATGTC |
| | ACACTGCAACGAGGCTCGGCTTATGGCGGCGTGTTATCTAATTTTTCCGGGACCGT |
| | AAAATATAGTGGCAGTAGCTATCCATTTCCGACCACCAGTGAAACGCCGCGGGTTG |
| | TTTATAATTCGAGAACGGATAAGCCGTGGCCGGTGGCGCTTTATTTGACGCCTGTG |
| | AGCAGTGCGGGCGGGGTGGTGATTAAAGCTGGCTCATTAATTGCCGTGCTTATTTT |
| | GCGACAGACCAACAACTATAACAGCGATGATTTCCAGTTTGTGTGGAATATTTACG |
| | CCAATAATGATGTGGTGGTGCCCACTGGCGGCTGCGATGTT |
| CH40-41 | fumC_allele 40 (SEQ ID NO: 12) |
| | CGAGCGCCATTCGGCAGGCGGCGGATGAAGTACTGGCAGGACAGCATGACGACGAA |
| | TTCCCGCTGGCTATCTGGCAGACCGGCTCCGGCACGCAAAGTAACATGAATATGAA |
| | CGAAGTGCTGGCTAACCGGGCCAGTGAATTACTCGGTGGCGTGCGCGGGATGGAAC |
| | GTAAAGTTCACCCTAACGACGACGTGAACAAAAGCCAAAGTTCCAACGATGTCTTT |
| | CCGACGGCGATGCACGTTGCGGCACTACTGGCGCTGCGCAAGCAACTCATTCCACA |
| | ACTTAAAACCCTGACCCAGACGCTGAGTGAAAAATCCCGCGCATTTGCTGATATCG |
| | TCAAAATCGGTCGTACCCACTTGCAGGACGCCACGCCGTTAACGCTGGGGCAGGAG |
| | ATTTCCGGCTGGGTAGCGATGCTGGAGCATAATCTCAAACATATCGAATACAGCCT |
| | GCCTCACGTAGCGGAACTGGC |
| | fimH_allele 41 (SEQ ID NO: 20) |
| | TTCGCCTGTAAAACCGCCAATGGTACAGCTATCCCTATTGGCGGTGGCAGCGCTAA |
| | TGTTTATGTAAACCTTGCGCCCGCCGTGAATGTGGGGCAAAACCTGGTCGTAGATC |
| | TTTCGACGCAAATCTTTTGCCATAACGATTATCCGGAAACCATTACAGACTATGTC |
| | ACACTGCAACGAGGCTCGGCTTATGGCGGCGTGTTATCTAATTTTTCCGGGACCGT |
| | AAAATATAGTGGCAGTAGCTATCCATTTCCTACCACCAGCGAAACGCCGCGCGTTG |
| | TTTATAATTCGAGAACGGATAAGCCGTGGCCGGTGGCGCTTTATTTGACGCCTGTG |
| | AGCAGTGCGGGCGGGGTGGCGATTAAAGCTGGCTCATTAATTGCCGTGCTTATTTT |
| | GCGACAGACCAACAACTATAACAGCGATGATTTCCAGTTTGTGTGGAATATTTACG |
| | CCAATAATGATGTGGTGGTGCCCACTGGCGGCTGTGATGTT |
| CH35-27 | fumC_allele 35 (SEQ ID NO: 21) |
| | CGAGCGCCATTCGGCAGGCGGCGGATGAAGTACTGGCAGGACAGCATGACGACGAA |
| | TTCCCATTGGCTATCTGGCAGACTGGCTCCGGCACGCAAAGTAACATGAACATGAA |
| | CGAAGTGCTGGCTAACCGGGCCAGTGAATTACTCGGCGGCGTGCGTGGGATGGAGC |
| | GTAAAGTTCACCCTAACGACGACGTGAACAAAAGCCAAAGTTCTAACGATGTCTTT |
| | CCAACGGCGATGCACGTTGCGGCGCTGCTGGCGCTGCGCAAGCAACTCATTCCGCA |
| | GCTTAAAACCCTGACACAGACGCTGAGTGAAAAATCGCGTGCATTTGCCGATATCG |
| | TAAAAATCGGTCGAACCCACTTGCAGGACGCCACGCCGCTAACACTGGGGCAGGAG |
| | ATTTCCGGCTGGGTAGCGATGCTCGAGCATAATCTCAAACATATTGAATACAGCCT |
| | GCCTCACGTAGCGGAACTGGC |
| | fimH_allele 27 (SEQ ID NO: 15) |
| | TTCGCCTGTAAAACCGCCAATGGTACCGCTATCCCTATTGGCGGTGGCAGCGCCAA |
| | TGTTTATGTAAACCTTGCGCCCGTCGTGAATGTGGGGCAAAACCTGGTCGTGGATC |
| | TTTCGACGCAAATCTTTTGCCATAACGATTATCCGGAAACCATTACAGACTATGTC |
| | ACACTGCAACGAGGCTCGGCTTATGGCGGCGTGTTATCTAATTTTTCCGGGACCGT |
| | AAAATATAGTGGCAGTAGCTATCCATTTCCTACCACCAGCGAAACGCCGCGCGTTG |
| | TTTATAATTCGAGAACGGATAAGCCGTGGCCGGTGGCGCTTTATTTGACGCCTGTG |
| | AGCAGTGCGGGCGGGGTGGCGATTAAAGCTGGCTCATTAATTGCCGTGCTTATTTT |
| | GCGACAGACCAACAACTATAACAGCGATGATTTCCAGTTTGTGTGGAATATTTACG |
| | CCAATAATGATGTGGTGGTGCCTACTGGCGGCTGCGATGTT |
| CH13-5 | fumC_allele 13 (SEQ ID NO: 22) |
| | CGAGCGCCATTCGGCAGGCGGCGGATGAAGTACTGGCAGGACAGCATGACGACGAA |
| | TTCCCGCTGGCTATCTGGCAGACCGGCTCCGGCACGCAAAGTAACATGAACATGAA |
| | CGAAGTGTTGGCTAACCGGGCCAGTGAATTACTCGGCGGCGTGCGCGGGATGGAAC |
| | GTAAAGTTCACCCTAACGACGACGTGAACAAAAGCCAAAGTTCCAACGATGTCTTT |
| | CCGACGGCGATGCACGTTGCGGCACTACTGGCGCTGCGCAAGCAACTCATTCCACA |
| | ACTTAAAACCCTGACCCAGACGCTGAGTGAAAAATCCCGCGCATTTGCCGATATCG |
| | TCAAAATCGGTCGTACCCACTTGCAGGACGCGACGCCGTTAACACTGGGGCAGGAG |
| | ATTTCCGGCTGGGTAGCGATGCTGGAGCATAATCTCAAACATATCGAATACAGCCT |
| | GCCTCACGTAACGGAACTGGC |
| | fimH_allele5 (SEQ ID NO: 17) |
| | TTCGCCTGTAAAACCGCCAATGGTACCGCTATCCCTATTGGCGGTGGCAGCGCCAA |
| | TGTTTATGTAAACCTTGCGCCTGCCGTGAATGTGGGGCAAAACCTGGTCGTGGATC |
| | TTTCGACGCAAATCTTTTGCCATAACGATTACCCGGAAACCATTACAGACTATGTC |
| | ACACTGCAACGAGGTTCGGCTTATGGCGGCGTGTTATCTAGTTTTTCCGGGACCGT |
| | AAAATATAATGGCAGTAGCTATCCTTTCCCTACTACCAGCGAAACGCCGCGGGTTG |
| | TTTATAATTCGAGAACGGATAAGCCGTGGCCGGTGGCGCTTTATTTGACGCCTGTG |
| | AGCAGTGCGGGGGGAGTGGCGATTAAAGCAGGCTCATTAATTGCCGTGCTTATTTT |
| | GCGACAGACCAACAACTATAACAGCGATGATTTCCAGTTTGTGTGGAATATTTACG |
| | CCAATAATGATGTGGTGGTGCCCACTGGCGGCTGCGATGTT |
| CH24-10 | fumC_allele 24 (SEQ ID NO: 23) |
| | CGAGCGCCATTCGGCAGGCGGCGGATGAAGTACTGGCAGGACAGCATGACGACGAA |
| | TTCCCGTTGGCTATCTGGCAGACCGGCTCCGGCACGCAAAGTAACATGAACATGAA |
| | CGAAGTGCTGGCTAACCGGGCCAGTGAATTACTCGGCGGCGTGCGCGGGATGGAAC |
| | GTAAAGTTCACCCTAACGACGACGTGAACAAAAGCCAAAGTTCCAACGATGTCTTT |
| | CCGACGGCGATGCACGTTGCGGCGCTGCTGGCGCTGCGCAAGCAACTCATTCCACA |
| | ACTTAAAACCCTGACCCAGACGCTGAGTGAAAAATCCCGCGCATTTGCCGATATCG |
| | TCAAAATCGGTCGTACCCACTTGCAGGACGCCACGCCGTTAACGCTGGGGCAGGAG |
| | ATTTCCGGCTGGGTAGCGATGCTGGAGCATAATCTCAAACATATCGAATACAGCCT |
| | GCCTCACGTAGCGGAACTGGC |
| | fimH_allele 10 (SEQ ID NO: 24) |
| | TTCGCCTGTAAAACCGCCAATGGTACCGCAATCCCTATTGGCGGTGGCAGCGCCAA |
| | TGTTTATGTAAACCTTGCGCCTGCCGTGAATGTGGGGCAAAACCTGGTCGTAGATC |
| | TTTCGACGCAAATCTTTTGCCATAACGATTACCCAGAAACCATTACAGACTATGTC |
| | ACACTGCAACGAGGTTCGGCTTATGGCGGCGTGTTATCTAGTTTTTCCGGGACCGT |
| | AAAATATAATGGCAGTAGCTATCCTTTCCCTACTACCAGCGAAACGCCGCGGGTTG |
| | TTTATAATTCGAGAACGGATAAGCCGTGGCCGGTGGCGCTTTATTTGACGCCGGTG |
| | AGCAGTGCGGGGGGAGTGGCGATTAAAGCTGGCTCATTAATTGCCGTGCTTATTTT |
| | GCGACAGACCAACAACTATAACAGCGATGATTTCCAGTTTGTGTGGAATATTTACG |
| | CCAATAATGATGTGGTGGTGCCCACTGGCGGCTGTGATGCT |
| CH40-22 | fumC_allele 40 (SEQ ID NO: 12) |
| | CGAGCGCCATTCGGCAGGCGGCGGATGAAGTACTGGCAGGACAGCATGACGACGAA |
| | TTCCCGCTGGCTATCTGGCAGACCGGCTCCGGCACGCAAAGTAACATGAATATGAA |
| | CGAAGTGCTGGCTAACCGGGCCAGTGAATTACTCGGTGGCGTGCGCGGGATGGAAC |
| | GTAAAGTTCACCCTAACGACGACGTGAACAAAAGCCAAAGTTCCAACGATGTCTTT |
| | CCGACGGCGATGCACGTTGCGGCACTACTGGCGCTGCGCAAGCAACTCATTCCACA |
| | ACTTAAAACCCTGACCCAGACGCTGAGTGAAAAATCCCGCGCATTTGCTGATATCG |
| | TCAAAATCGGTCGTACCCACTTGCAGGACGCCACGCCGTTAACGCTGGGGCAGGAG |
| | ATTTCCGGCTGGGTAGCGATGCTGGAGCATAATCTCAAACATATCGAATACAGCCT |
| | GCCTCACGTAGCGGAACTGGC |
| | fimH_allele 22 (SEQ ID NO: 25) |
| | TTCGCCTGTAAAACCGCCAATGGTACCGCAATCCCTATTGGCGGTGGCAGCGCCAA |
| | TGTTTATGTAAACCTTGCGCCTGCCGTGAATGTGGGGCAAAACCTGGTCGTGGATC |
| | TTTCGACGCAAATCTTTTGCCATAACGATTACCCGGAAACCATTACAGATTATGTC |
| | ACACTGCAACGAGGCTCGGCTTATGGCGGCGTGTTATCTAATTTTTCCGGGACCGT |
| | AAAATATAATGGCAGTAGCTATCCTTTCCCTACTACCAGCGAAACGCCGCGGGTTG |
| | TTTATAATTCGAGAACGGATAAGCCGTGGCCGGTGGCGCTTTATTTGACGCCTGTG |
| | AGCAGTGCGGGGGGAGTGGCGATTAAAGCTGGCTCATTAATTGCCGTGCTAATTTT |
| | GCGACAGACCAACAACTATAACAGCGATGATTTCCAGTTTGTGTGGAATATTTACG |
| | CCAATAATGATGTGGTGGTGCCTACTGGCGGCTGTGATGTT |
| CH14-27 | fumC_allele 14 (SEQ ID NO: 26) |
| | CGAGCGCCATTCGGCAGGCGGCGGATGAAGTACTGGCAGGACAGCATGACGACGAA |
| | TTCCCGCTGGCTATCTGGCAGACCGGCTCCGGCACGCAAAGTAACATGAACATGAA |
| | TGAAGTGCTGGCTAACCGGGCCAGTGAATTACTCGGCGGCGTGCGCGGGATGGAAC |
| | GTAAAGTTCACCCTAACGACGACGTGAACAAAAGCCAAAGTTCCAACGATGTCTTT |
| | CCGACGGCGATGCACGTTGCGGCACTACTGGCGCTGCGCAAGCAACTCATTCCACA |
| | ACTTAAAACCCTGACCCAGACGCTGAGTGAAAAATCCCGCGCATTTGCCGATATCG |
| | TCAAAATCGGTCGTACCCACTTGCAGGACGCGACGCCGTTAACGCTGGGGCAGGAG |
| | ATTTCCGGCTGGGTAGCGATGCTGGAGCATAATCTCAAACATATCGAATACAGCCT |
| | GCCTCACGTAGCGGAACTGGC |
| | fimH_allele 27 (SEQ ID NO: 15) |
| | TTCGCCTGTAAAACCGCCAATGGTACCGCTATCCCTATTGGCGGTGGCAGCGCCAA |
| | TGTTTATGTAAACCTTGCGCCCGTCGTGAATGTGGGGCAAAACCTGGTCGTGGATC |
| | TTTCGACGCAAATCTTTTGCCATAACGATTATCCGGAAACCATTACAGACTATGTC |
| | ACACTGCAACGAGGCTCGGCTTATGGCGGCGTGTTATCTAATTTTTCCGGGACCGT |
| | AAAATATAGTGGCAGTAGCTATCCATTTCCTACCACCAGCGAAACGCCGCGCGTTG |
| | TTTATAATTCGAGAACGGATAAGCCGTGGCCGGTGGCGCTTTATTTGACGCCTGTG |
| | AGCAGTGCGGGCGGGGTGGCGATTAAAGCTGGCTCATTAATTGCCGTGCTTATTTT |
| | GCGACAGACCAACAACTATAACAGCGATGATTTCCAGTTTGTGTGGAATATTTACG |
| | CCAATAATGATGTGGTGGTGCCTACTGGCGGCTGCGATGTT |
| CH24-30 | fumC_allele 24 (SEQ ID NO: 23) |
| | CGAGCGCCATTCGGCAGGCGGCGGATGAAGTACTGGCAGGACAGCATGACGACGAA |
| | TTCCCGTTGGCTATCTGGCAGACCGGCTCCGGCACGCAAAGTAACATGAACATGAA |
| | CGAAGTGCTGGCTAACCGGGCCAGTGAATTACTCGGCGGCGTGCGCGGGATGGAAC |
| | GTAAAGTTCACCCTAACGACGACGTGAACAAAAGCCAAAGTTCCAACGATGTCTTT |
| | CCGACGGCGATGCACGTTGCGGCGCTGCTGGCGCTGCGCAAGCAACTCATTCCACA |
| | ACTTAAAACCCTGACCCAGACGCTGAGTGAAAAATCCCGCGCATTTGCCGATATCG |
| | TCAAAATCGGTCGTACCCACTTGCAGGACGCCACGCCGTTAACGCTGGGGCAGGAG |
| | ATTTCCGGCTGGGTAGCGATGCTGGAGCATAATCTCAAACATATCGAATACAGCCT |
| | GCCTCACGTAGCGGAACTGGC |
| | fimH_allele 30 (SEQ ID NO: 13) |
| | TTCGCCTGTAAAACCGCCAATGGTACCGCTATTCCTATTGGCGGTGGCAGCGCTAA |
| | TGTTTATGTAAACCTTGCGCCTGCCGTGAATGTGGGGCAAAACCTGGTCGTAGATC |
| | TTTCGACGCAAATCTTTTGCCATAACGATTATCCGGAAACCATTACAGACTATGTC |
| | ACACTGCAACGAGGCTCGGCTTATGGCGGCGTGTTATCTAATTTTTCCGGGACCGT |
| | AAAATATAGTGGCAGTAGCTATCCATTTCCGACTACCAGCGAAACGCCGCGGGTTG |
| | TTTATAATTCGAGAACGGATAAGCCGTGGCCGGTGGCGCTTTATTTGACGCCTGTG |
| | AGCAGTGCGGGTGGGGTGGCGATTAAAGCTGGCTCATTAATTGCCGTGCTTATTTT |
| | GCGACAGACCAACAACTATAACAGCGATGATTTCCAGTTTGTGTGGAATATTTACG |
| | CCAATAATGATGTGGTGGTGCCTACTGGCGGCTGCGATGTT |
| CH38-27 | fumC_allele 38 (SEQ ID NO: 27) |
| | CGAGCGCCATTCGGCAGGCGGCGGATGAAGTACTGGCAGGACAGCATGACGACGAA |
| | TTCCCGCTGGCTATCTGGCAGACCGGCTCCGGCACGCAAAGTAACATGAACATGAA |
| | CGAAGTGCTGGCTAACCGGGCCAGTGAATTACTCGGCGGCGTGCGCGGGATGGAAC |
| | GTAAAGTTCACCCTAACGACGACGTGAACAAAAGCCAAAGTTCCAACGATGTCTTT |
| | CCGACGGCGATGCACGTTGCGGCGCTGCTGGCGCTGCGCAAGCAACTCATTCCACA |
| | ACTTAAAACCCTGACCCAGACGCTGAGTGAAAAATCCCGCGCATTTGCCGATATCG |
| | TCAAAATCGGTCGTACCCACTTGCAGGACGCCACGCCGTTAACGCTGGGGCAGGAG |
| | ATTTCCGGCTGGGTAGCGATGCTGGAGCATAATCTCAAACATATCGAATACAGCCT |
| | GCCTCACGTAGCGGAACTGGC |
| | fimH_allele 27 (SEQ ID NO: 15) |
| | TTCGCCTGTAAAACCGCCAATGGTACCGCTATCCCTATTGGCGGTGGCAGCGCCAA |
| | TGTTTATGTAAACCTTGCGCCCGTCGTGAATGTGGGGCAAAACCTGGTCGTGGATC |
| | TTTCGACGCAAATCTTTTGCCATAACGATTATCCGGAAACCATTACAGACTATGTC |
| | ACACTGCAACGAGGCTCGGCTTATGGCGGCGTGTTATCTAATTTTTCCGGGACCGT |
| | AAAATATAGTGGCAGTAGCTATCCATTTCCTACCACCAGCGAAACGCCGCGCGTTG |
| | TTTATAATTCGAGAACGGATAAGCCGTGGCCGGTGGCGCTTTATTTGACGCCTGTG |
| | AGCAGTGCGGGCGGGGTGGCGATTAAAGCTGGCTCATTAATTGCCGTGCTTATTTT |
| | GCGACAGACCAACAACTATAACAGCGATGATTTCCAGTTTGTGTGGAATATTTACG |
| | CCAATAATGATGTGGTGGTGCCTACTGGCGGCTGCGATGTT |
| CH40-20 | fumC_allele 40 (SEQ ID NO: 12) |
| | CGAGCGCCATTCGGCAGGCGGCGGATGAAGTACTGGCAGGACAGCATGACGACGAA |
| | TTCCCGCTGGCTATCTGGCAGACCGGCTCCGGCACGCAAAGTAACATGAATATGAA |
| | CGAAGTGCTGGCTAACCGGGCCAGTGAATTACTCGGTGGCGTGCGCGGGATGGAAC |
| | GTAAAGTTCACCCTAACGACGACGTGAACAAAAGCCAAAGTTCCAACGATGTCTTT |
| | CCGACGGCGATGCACGTTGCGGCACTACTGGCGCTGCGCAAGCAACTCATTCCACA |
| | ACTTAAAACCCTGACCCAGACGCTGAGTGAAAAATCCCGCGCATTTGCTGATATCG |
| | TCAAAATCGGTCGTACCCACTTGCAGGACGCCACGCCGTTAACGCTGGGGCAGGAG |
| | ATTTCCGGCTGGGTAGCGATGCTGGAGCATAATCTCAAACATATCGAATACAGCCT |
| | GCCTCACGTAGCGGAACTGGC |
| | fimH_allele 20 (SEQ ID NO: 28) |
| | TTCGCCTGTAAAACCGCCAATGGTACCGCAATCCCTATTGGCGGTGGCAGCGCCAA |
| | TGTTTATGTAAACCTTGCGCCTGCCGTGAATGTGGGGCAAAACCTGGTCGTGGATC |
| | TTTCGACGCAAATCTTTTGCCATAACGATTACCCGGAAACCATTACAGATTATGTC |
| | ACACTGCAACGAGGCTCGGCTTATGGTGGCGTGTTATCTAATTTTTCCGGGACCGT |
| | AAAATATAATGGCAGTAGCTATCCTTTCCCTACTACCAGCGAAACGCCGCGGGTTG |
| | TTTATAATTCGAGAACGGATAAGCCGTGGCCGGTGGCGCTTTATTTGACGCCTGTG |
| | AGCAGTGCGGGGGGAGTGGCGATTAAAGCTGGCTCATTAATTGCCGTGCTTATTTT |
| | GCGACAGACCAACAACTATAACAGCGATGATTTCCAGTTTGTGTGGAATATTTACG |
| | CCAATAATGATGTGGTGGTGCCCACTGGCGGCTGCGATGTT |
| CH24-9 | fumC_allele 24 (SEQ ID NO: 23) |
| | CGAGCGCCATTCGGCAGGCGGCGGATGAAGTACTGGCAGGACAGCATGACGACGAA |
| | TTCCCGTTGGCTATCTGGCAGACCGGCTCCGGCACGCAAAGTAACATGAACATGAA |
| | CGAAGTGCTGGCTAACCGGGCCAGTGAATTACTCGGCGGCGTGCGCGGGATGGAAC |
| | GTAAAGTTCACCCTAACGACGACGTGAACAAAAGCCAAAGTTCCAACGATGTCTTT |
| | CCGACGGCGATGCACGTTGCGGCGCTGCTGGCGCTGCGCAAGCAACTCATTCCACA |
| | ACTTAAAACCCTGACCCAGACGCTGAGTGAAAAATCCCGCGCATTTGCCGATATCG |
| | TCAAAATCGGTCGTACCCACTTGCAGGACGCCACGCCGTTAACGCTGGGGCAGGAG |
| | ATTTCCGGCTGGGTAGCGATGCTGGAGCATAATCTCAAACATATCGAATACAGCCT |
| | GCCTCACGTAGCGGAACTGGC |
| | fimH_allele 9 (SEQ ID NO: 29) |
| | TTCGCCTGTAAAACCGCCAATGGTACCGCAATCCCTATTGGCGGTGGCAGCGCCAA |
| | TGTTTATGTAAACCTTGCGCCTGCCGTGAATGTGGGGCAAAACCTGGTCGTAGATC |
| | TTTCGACGCAAATCTTTTGCCATAACGATTACCCAGAAACCATTACAGACTATGTC |
| | ACACTGCAACGAGGTTCGGCTTATGGCGGCGTGTTATCTAGTTTTTCCGGGACCGT |
| | AAAATATAATGGCAGTAGCTATCCTTTCCCTACTACCAGCGAAACGCCGCGGGTTG |
| | TTTATAATTCGAGAACGGATAAGCCGTGGCCGGTGGCGCTTTATTTGACGCCGGTG |
| | AGCAGTGCGGGGGGAGTGGCGATTAAAGCTGGCTCATTAATTGCCGTGCTTATTTT |
| | GCGACAGACCAACAACTATAACAGCGATGATTTCCAGTTTGTGTGGAATATTTACG |
| | CCAATAATGATGTGGTGGTGCCCACTGGCGGCTGTGATGTT |
| CH14-2 | fumC_allele 14 (SEQ ID NO: 26) |
| | CGAGCGCCATTCGGCAGGCGGCGGATGAAGTACTGGCAGGACAGCATGACGACGAA |
| | TTCCCGCTGGCTATCTGGCAGACCGGCTCCGGCACGCAAAGTAACATGAACATGAA |
| | TGAAGTGCTGGCTAACCGGGCCAGTGAATTACTCGGCGGCGTGCGCGGGATGGAAC |
| | GTAAAGTTCACCCTAACGACGACGTGAACAAAAGCCAAAGTTCCAACGATGTCTTT |
| | CCGACGGCGATGCACGTTGCGGCACTACTGGCGCTGCGCAAGCAACTCATTCCACA |
| | ACTTAAAACCCTGACCCAGACGCTGAGTGAAAAATCCCGCGCATTTGCCGATATCG |
| | TCAAAATCGGTCGTACCCACTTGCAGGACGCGACGCCGTTAACGCTGGGGCAGGAG |
| | ATTTCCGGCTGGGTAGCGATGCTGGAGCATAATCTCAAACATATCGAATACAGCCT |
| | GCCTCACGTAGCGGAACTGGC |
| | fimH_allele 2 (SEQ ID NO: 30) |
| | TTCGCCTGTAAAACCGCCAATGGTACCGCTATCCCTATTGGCGGTGGCAGCGCCAA |
| | TGTTTATGTAAACCTTGCGCCTGCCGTGAATGTGGGGCAAAACCTGGTCGTGGATC |
| | TTTCGACGCAAATCTTTTGCCATAACGATTACCCGGAAACCATTACAGACTATGTC |
| | ACACTGCAACGAGGTTCGGCTTATGGCGGCGTGTTATCTAGTTTTTCCGGGACCGT |
| | AAAATATAATGGCAGTAGCTATCCTTTCCCTACTACCAGCGAAACGCCGCGGGTTG |
| | TTTATAATTCGAGAACGGATAAGCCGTGGCCGGTGGCGCTTTATTTGACGCCTGTG |
| | AGCAGTGCGGGGGGAGTGGCGATTAAAGCTGGCTCATTAATTGCCGTGCTTATTTT |
| | GCGACAGACCAACAACTATAACAGCGATGATTTCCAGTTTGTGTGGAATATTTACG |
| | CCAATAATGATGTGGTGGTGCCCACTGGCGGCTGTGATGTT |
| CH38-5 | fumC_allele 38 (SEQ ID NO: 27) |
| | CGAGCGCCATTCGGCAGGCGGCGGATGAAGTACTGGCAGGACAGCATGACGACGAA |
| | TTCCCGCTGGCTATCTGGCAGACCGGCTCCGGCACGCAAAGTAACATGAACATGAA |
| | CGAAGTGCTGGCTAACCGGGCCAGTGAATTACTCGGCGGCGTGCGCGGGATGGAAC |
| | GTAAAGTTCACCCTAACGACGACGTGAACAAAAGCCAAAGTTCCAACGATGTCTTT |
| | CCGACGGCGATGCACGTTGCGGCGCTGCTGGCGCTGCGCAAGCAACTCATTCCACA |
| | ACTTAAAACCCTGACCCAGACGCTGAGTGAAAAATCCCGCGCATTTGCCGATATCG |
| | TCAAAATCGGTCGTACCCACTTGCAGGACGCCACGCCGTTAACGCTGGGGCAGGAG |
| | ATTTCCGGCTGGGTAGCGATGCTGGAGCATAATCTCAAACATATCGAATACAGCCT |
| | GCCTCACGTAGCGGAACTGGC |
| | fimH_allele5 (SEQ ID NO: 17) |
| | TTCGCCTGTAAAACCGCCAATGGTACCGCTATCCCTATTGGCGGTGGCAGCGCCAA |
| | TGTTTATGTAAACCTTGCGCCTGCCGTGAATGTGGGGCAAAACCTGGTCGTGGATC |
| | TTTCGACGCAAATCTTTTGCCATAACGATTACCCGGAAACCATTACAGACTATGTC |
| | ACACTGCAACGAGGTTCGGCTTATGGCGGCGTGTTATCTAGTTTTTCCGGGACCGT |
| | AAAATATAATGGCAGTAGCTATCCTTTCCCTACTACCAGCGAAACGCCGCGGGTTG |
| | TTTATAATTCGAGAACGGATAAGCCGTGGCCGGTGGCGCTTTATTTGACGCCTGTG |
| | AGCAGTGCGGGGGGAGTGGCGATTAAAGCAGGCTCATTAATTGCCGTGCTTATTTT |
| | GCGACAGACCAACAACTATAACAGCGATGATTTCCAGTTTGTGTGGAATATTTACG |
| | CCAATAATGATGTGGTGGTGCCCACTGGCGGCTGCGATGTT |
| CH38-41 | fumC_allele 38 (SEQ ID NO: 27) |
| | CGAGCGCCATTCGGCAGGCGGCGGATGAAGTACTGGCAGGACAGCATGACGACGAA |
| | TTCCCGCTGGCTATCTGGCAGACCGGCTCCGGCACGCAAAGTAACATGAACATGAA |
| | CGAAGTGCTGGCTAACCGGGCCAGTGAATTACTCGGCGGCGTGCGCGGGATGGAAC |
| | GTAAAGTTCACCCTAACGACGACGTGAACAAAAGCCAAAGTTCCAACGATGTCTTT |
| | CCGACGGCGATGCACGTTGCGGCGCTGCTGGCGCTGCGCAAGCAACTCATTCCACA |
| | ACTTAAAACCCTGACCCAGACGCTGAGTGAAAAATCCCGCGCATTTGCCGATATCG |
| | TCAAAATCGGTCGTACCCACTTGCAGGACGCCACGCCGTTAACGCTGGGGCAGGAG |
| | ATTTCCGGCTGGGTAGCGATGCTGGAGCATAATCTCAAACATATCGAATACAGCCT |
| | GCCTCACGTAGCGGAACTGGC |
| | fimH_allele 41 (SEQ ID NO: 20) |
| | TTCGCCTGTAAAACCGCCAATGGTACAGCTATCCCTATTGGCGGTGGCAGCGCTAA |
| | TGTTTATGTAAACCTTGCGCCCGCCGTGAATGTGGGGCAAAACCTGGTCGTAGATC |
| | TTTCGACGCAAATCTTTTGCCATAACGATTATCCGGAAACCATTACAGACTATGTC |
| | ACACTGCAACGAGGCTCGGCTTATGGCGGCGTGTTATCTAATTTTTCCGGGACCGT |
| | AAAATATAGTGGCAGTAGCTATCCATTTCCTACCACCAGCGAAACGCCGCGCGTTG |
| | TTTATAATTCGAGAACGGATAAGCCGTGGCCGGTGGCGCTTTATTTGACGCCTGTG |
| | AGCAGTGCGGGCGGGGTGGCGATTAAAGCTGGCTCATTAATTGCCGTGCTTATTTT |
| | GCGACAGACCAACAACTATAACAGCGATGATTTCCAGTTTGTGTGGAATATTTACG |
| | CCAATAATGATGTGGTGGTGCCCACTGGCGGCTGTGATGTT |
| CH38-15 | fumC_allele 38 (SEQ ID NO: 27) |
| | CGAGCGCCATTCGGCAGGCGGCGGATGAAGTACTGGCAGGACAGCATGACGACGAA |
| | TTCCCGCTGGCTATCTGGCAGACCGGCTCCGGCACGCAAAGTAACATGAACATGAA |
| | CGAAGTGCTGGCTAACCGGGCCAGTGAATTACTCGGCGGCGTGCGCGGGATGGAAC |
| | GTAAAGTTCACCCTAACGACGACGTGAACAAAAGCCAAAGTTCCAACGATGTCTTT |
| | CCGACGGCGATGCACGTTGCGGCGCTGCTGGCGCTGCGCAAGCAACTCATTCCACA |
| | ACTTAAAACCCTGACCCAGACGCTGAGTGAAAAATCCCGCGCATTTGCCGATATCG |
| | TCAAAATCGGTCGTACCCACTTGCAGGACGCCACGCCGTTAACGCTGGGGCAGGAG |
| | ATTTCCGGCTGGGTAGCGATGCTGGAGCATAATCTCAAACATATCGAATACAGCCT |
| | GCCTCACGTAGCGGAACTGGC |
| | fimH_allele 15 (SEQ ID NO: 31) |
| | TTCGCCTGTAAAACCGCCAATGGTACCGCAATCCCTATTGGCGGTGGCAGCGCCAA |
| | TGTTTATGTAAACCTTGCGCCTGCCGTGAATGTGGGGCAAAACCTGGTCGTAGATC |
| | TTTCGACGCAAATCTTTTGCCATAACGATTACCCAGAAACCATTACAGACTATGTC |
| | ACACTGCAACGAGGTTCGGCTTATGGCGGCGTGTTATCTAGTTTTTCCGGGACCGT |
| | AAAATATAATGGCAGTAGCTATCCTTTCCCTACTACCAGCGAAACGCCGCGGGTTG |
| | TTTATAATTCGAGAACGGATAAGCCGTGGCCGGTGGCGCTTTATTTGACGCCGGTG |
| | AGCAGTGCGGGGGGAGTGGCGATTAAAGCTGGCTCATTAATTGCCGTGCTTATTTT |
| | GCGACAGACCAACAACTATAACAGCGATGATTTCCAGTTTGTGTGGAATATTTACG |
| | CCAATAATGATGTGGTGGTGCCCACTGGCGGCTGCGATGTT |
| CH52-14 | fumC_allele 52 (SEQ ID NO: 32) |
| | CGAGCGCCATTCGGCAGGCGGCGGATGAAGTACTGGCAGGACAGCATGACGACGAA |
| | TTCCCGCTGGCTATCTGGCAGACCGGCTCCGGCACGCAAAGTAACATGAACATGAA |
| | CGAAGTGCTGGCTAACCGGGCCAGTGAATTACTCGGTGGCGTGCGCGGGATGGAAC |
| | GTAAAGTTCACCCTAACGACGACGTGAACAAAAGCCAAAGTTCCAACGATGTCTTT |
| | CCGACGGCGATGCACGTTGCGGCACTACTGGCGCTGCGCAAGCAACTCATTCCACA |
| | ACTTAAAACCCTGACCCAGACGCTGAGTGAAAAATCCCGCGCATTTGCTGATATCG |
| | TCAAAATCGGTCGTACCCACTTGCAGGACGCGACGCCGTTAACGCTGGGGCAGGAG |
| | ATTTCCGGCTGGGTAGCGATGCTGGAGCATAATCTCAAACATATCGAATACAGCCT |
| | GCCTCACGTAGCGGAACTGGC |
| | fimH_allele 14 (SEQ ID NO: 33) |
| | TTCGCCTGTAAAACCGCCAATGGTACCGCAATCCCTATTGGCGGTGGCAGCGCCAA |
| | TGTTTATGTAAACCTTGCGCCTGCCGTGAATGTGGGGCAAAACCTGGTCGTGGATC |
| | TTTCGACGCAAATCTTTTGCCATAACGATTACCCGGAAACCATTACAGACTATGTC |
| | ACACTGCAACGAGGTTCGGCTTATGGCGGCGTGTTATCTAGTTTTTCCGGGACCGT |
| | AAAATATAATGGCAGTAGCTATCCTTTCCCTACTACCAGCGAAACGCCGCGGGTTG |
| | TTTATAATTCGAGAACGGATAAGCCGTGGCCGGTGGCGCTTTATTTGACGCCGGTG |
| | AGCAGTGCGGGGGGAGTGGCGATTAAAGCTGGCTCATTAATTGCCGTGCTTATTTT |
| | GCGACAGACCAACAACTATAACAGCGATGATTTCCAGTTTGTGTGGAATATTTACG |
| | CCAATAATGATGTGGTGGTGCCCACTGGCGGCTGTGATGTT |

In additional embodiments, the typing indicates efficacy of an antibiotic treatment. For example, the CH40-30, CH4-27, CH11-54, CH13-5, CH40-22 and CH35-27 clonotypes are resistant to certain antibiotic treatments (e.g. AMP, TET, A/S, T/S, A/K, CEF or CIP), while the CH38-41, CH38-15, CH52-14, CH24-9 and CH14-2 clonotypes are susceptible to certain antibiotic treatments (e.g., AMP, TET, A/S, T/S, A/K, CEF or CIP). In such an example, if clonotype CH40-30 was identified as being resistant to a specific antibiotic (e.g., AMP), then ampicillin would not be considered to be effective in treating the E. coli clonotype CH40-30 with an antibiotic treatment comprising ampicillin. In another example, if clonotype CH38-41 was identified as being sensitive to a specific antibiotic (e.g., AMP), then ampicillin would be considered to be effective in treating the E. coli clonotype CH38-41 with an antibiotic treatment comprising ampicillin. Antibiotic or antimicrobial treatments or therapies include any agent, or combination of agents, that selectively kills or inhibits the growth of E. coli. Antibiotics may include, but are not limited to ampicillin (AMP), tetracycline (TET), ampicillin-sulbactam (A/S), trimethoprimsulfamethoxazole (T/S), amoxicillin-clavulanate (A/K), cefazolin (CZ), ciprofloxacin (CIP), gentamicin (GM), nitrofurantoin (NIT), ceftriaxone (CTR) and/or piperacillin-tazobactam (PTZ).

In one embodiment of the invention, the typing is carried out after the subject has undergone treatment for the disease state or the infection with antibiotic resistant E. coli, and the typing indicates efficacy of the treatment. The methods of the invention may be used to identify what clonotypes are present after treatment. This could determine, for example, if the treatment eradicated the disease-causing E. coli; or if the antibiotic resistant E. coli are still present. In another embodiment, the methods of the invention could be used to identify a single strain, two strains or three strains etc., as causative or likely to be causative of a disease prior to treatment, during treatment or after treatment.

The nucleic acid sequence of any suitable portion of the fimH gene can be determined to carry out the methods of the invention. In one embodiment, the entirety of the fimH gene can be determined. In another embodiment, a portion of the fimH gene can be determined. In some embodiments, a fragment suitable for efficient molecular typing (<500 nt) may be used. The portion of fimH sequenced may include the nucleotides encoding mature peptide codons 1 to 163 corresponding to nucleotides 1-489 of reference sequence SEQ ID NO:04, which span the entire mannose binding lectin domain, the interdomain linker, and a few N-terminal residues of the pilin domain. In one embodiment, the portion of the fimH gene is amplified by an oligonucleotide primer pair consisting of 5′-CACTCAGGGAACCATTCAGGCA-3′ (SEQ ID NO: 01) and 5′-CTTATTGATAAACAAAAGTCAC-3′ (SEQ ID NO: 02).

In one embodiment, sequencing of the fumC gene is combined with fimH. In this embodiment, the nucleic acid sequence of any suitable portion of the fumC gene can be determined to carry out the methods of the invention. In one embodiment the entirety of the fumC gene locus can be determined. In another embodiment, a portion of the fumC gene locus can be determined. In some embodiments, a fragment of the fumC gene suitable for efficient molecular typing (<500 nt) may be used. In another embodiment, the portion of the fumC gene is amplified by an oligonucleotide primer pair specific for an ˜500 nt fragment, an ˜400 nt fragment, an ˜300 nt fragment, an ˜200 nt fragment or an ˜100 nt fragment as designed by person of ordinary skill in the art for efficient molecular typing. As shown in the examples below, the inventors discovered that pairing fumC with fimH provided the best ability to distinguish E. coli substrains. FumC was selected for pairing with fimH in this clonotyping scheme because, of the 7 MLST loci, it (i) provided the best discriminatory power when combined with fimH, (ii) exhibited the highest level of nucleotide polymorphism, and (iii) was best able to predict the phylogenetic group. This combination of fumC and fimH provided greater discriminatory power than standard 7-locus MLST, which over the past decade replaced multilocus enzyme electrophoresis as the standard method for studying E. coli population structure.

Surprisingly, clonotyping based on fumC and fimH(CH clonotype) identified specific STs or ST complexes for more than 90% of the isolates. CH clonotyping is applicable as a molecular tool for both applied and basic investigations regarding the epidemiology and population structure of E. coli. For example, CH clonotyping can be used to screen isolates in suspected point source outbreaks and to evaluate large clinical isolate collections for sub-ST clonal diversity (e.g., in population studies of antimicrobial resistance).

Furthermore, the inventors have discovered that the clonotypes of E. coli isolates, as inferred from fumC and fimH, are linked to distinct antimicrobial susceptibility profiles and clinical manifestations. These findings indicate that a clonotype-guided approach substantially reduces the likelihood of drug-bug mismatches during the course of initial antimicrobial therapy by providing more specific data about a patient's actual organism. Furthermore, if clonotyping profiles are made available in a timely fashion as part of clinical laboratory diagnostics, trimethoprim-sulfamethoxazole and/or fluoroquinolones can be used with higher confidence against the majority of clinical E. coli isolates, with projected averages of 3- and 5-fold reductions, respectively, in the likelihood of drug-bug mismatch compared with standard empirical use of the corresponding antimicrobials. Thus, greater certainty from the outset regarding which antimicrobials can and cannot be used reliably for a given patient with suspected E. coli infection will be of great benefit to patients and health care systems alike.

In another embodiment, sequencing of the adk gene is combined with fimH. In this embodiment, the entirety of the adk gene locus can be determined. In another embodiment, a portion of the adk gene locus can be determined. In some embodiments, a fragment of the adk gene suitable for efficient molecular typing (<500 nt) may be used.

In a further embodiment, sequencing of the gyrB gene is combined with fimH In this embodiment, the entirety of the gyrB gene locus can be determined. In another embodiment, a portion of the gyrB gene locus can be determined. In some embodiments, a fragment of the gyrB gene suitable for efficient molecular typing (<500 nt) may be used.

In yet another embodiment, sequencing of the icd gene is combined with fimH. In this embodiment, the entirety of the icd gene locus can be determined. In another embodiment, a portion of the icd gene locus can be determined. In some embodiments, a fragment of the icd gene suitable for efficient molecular typing (<500 nt) may be used.

In another embodiment, sequencing of the mdh gene is combined with fimH. In this embodiment, the entirety of the mdh gene locus can be determined. In another embodiment, a portion of the mdh gene locus can be determined. In some embodiments, a fragment of the mdh gene suitable for efficient molecular typing (<500 nt) may be used.

In another embodiment, sequencing of the purA gene is combined with fimH. In this embodiment, the entirety of the purA gene locus can be determined. In another embodiment, a portion of the purA gene locus can be determined. In some embodiments, a fragment of the purA gene suitable for efficient molecular typing (<500 nt) may be used.

In another embodiment, sequencing of the recA gene is combined with fimH. In this embodiment, the entirety of the recA gene locus can be determined. In another embodiment, a portion of the recA gene locus can be determined. In some embodiments, a fragment of the recA gene suitable for efficient molecular typing (<500 nt) may be used.

Any suitable amplification technique can be used, including but not limited to PCR, RT-PCT, qPCR, spPCR, etc. Suitable amplification conditions can be determined by those of skill in the art based on the particular primer pair design and other factors, based on the teachings herein.

Any suitable sequencing technique can be used, including but not limited to Sanger sequencing, Maxam-Gilbert sequencing, or any of the next generation sequencing methods (e.g., pryoseqeuncing (454); sequencing by synthesis (Illumina); ion torrent sequencing, single-molecule real-time sequencing or SOLiD sequencing etc. Suitable sequencing conditions can be determined by those of skill in the art based on the particular factors, based on the teachings herein.

In a second aspect, the invention provides a composition consisting of between 2-5 oligonucleotide primer pairs, wherein: (a) a first primer pair selectively amplifies a region of a fimH gene; and (b) a second primer pair selectively amplifies a region of a gene selected from the group consisting of fumC, adk, gyrB, icd, mdh, purA, and recA. In some embodiments of the second aspect of the invention, the first primer pair consists of 5′-CACTCAGGGAACCATTCAGGCA-3′ (SEQ ID NO: 01) and 5′-CTTATTGATAAACAAAAGTCAC-3′ (SEQ ID NO: 02).

In one embodiment of the second aspect of the invention, 2 primer pairs are used, 3 primer pairs are use, 4 primer pairs are used or 5 primer pairs are used. In this embodiment, primer pairs would be selected from oligonucleotides capable of amplifying the entirety or a portion of the fimH gene along with the entirety or a portion of at least one or more of the genes selected from the fumC, adk, gyrB, icd, mdh, purA, and recA.

In one embodiment of the second aspect, a primer pair for the fumC gene is combined with a primer pair for fimH. In this embodiment, the primer pair would sequence the entirety of the fumC gene locus. In another embodiment, a portion of the fumC gene locus can be determined by the fumC primer pair. In some embodiments, a primer pair specific for a fragment of the fumC gene suitable for efficient molecular typing (<500 nt) may be used. In another embodiment, the portion of the fumC gene is amplified by an oligonucleotide primer pair specific for an ˜500 nt fragment, an ˜400 nt fragment, an ˜300 nt fragment, an ˜200 nt fragment or an ˜100 nt fragment as designed by person of ordinary skill in the art for efficient molecular typing. As shown in the examples below, the inventors discovered that pairing fumC with fimH was the most suitable. FumC was selected as a housekeeping locus for pairing with fimH in this clonotyping scheme because, of the 7 MLST loci, it (i) provided the best discriminatory power, (ii) exhibited the highest level of nucleotide polymorphism, and (iii) was best able to predict the phylogenetic group. This combination of fumC and fimH provided greater discriminatory power than standard 7-locus MLST, which over the past decade replaced multilocus enzyme electrophoresis as the standard method for studying E. coli population structure.

In another embodiment of the second aspect of the invention, a primer pair for the adk gene is combined with the fimH primer pair. In this embodiment, the entirety of the adk gene locus can be determined by the primer pair. In another embodiment, a portion of the adk gene locus can be determined by the primer pair. In some embodiments, a fragment of the adk gene suitable for efficient molecular typing (<500 nt) may be used.

In a further embodiment of the second aspect of the invention, a primer pair for the gyrB gene is combined with the fimH primer pair. In this embodiment, the entirety of the gyrB gene locus can be determined by the primer pair. In another embodiment, a portion of the gyrB gene locus can be determined by the primer pair. In some embodiments, a fragment of the gyrB gene suitable for efficient molecular typing (<500 nt) may be used.

In yet another embodiment of the second aspect of the invention, a primer pair for the icd gene is combined with the fimH primer pair. In this embodiment, the entirety of the icd gene locus can be determined by the primer pair. In another embodiment, a portion of the icd gene locus can be determined by the primer pair. In some embodiments, a fragment of the icd gene suitable for efficient molecular typing (<500 nt) may be used.

In another embodiment of the second aspect of the invention, a primer pair for the mdh gene is combined with the fimH primer pair. In this embodiment, the entirety of the mdh gene locus can be determined by the primer pair. In another embodiment, a portion of the mdh gene locus can be determined by the primer pair. In some embodiments, a fragment of the mdh gene suitable for efficient molecular typing (<500 nt) may be used.

In another embodiment of the second aspect of the invention, a primer pair for the purA gene is combined with the fimH primer pair. In this embodiment, the entirety of the purA gene locus can be determined by the primer pair. In another embodiment, a portion of the purA gene locus can be determined by the primer pair. In some embodiments, a fragment of the purA gene suitable for efficient molecular typing (<500 nt) may be used.

In another embodiment of the second aspect of the invention, a primer pair for the recA gene is combined with the fimH primer pair. In this embodiment, the entirety of the recA gene locus can be determined by the primer pair. In another embodiment, a portion of the recA gene locus can be determined by the primer pair. In some embodiments, a fragment of the recA gene suitable for efficient molecular typing (<500 nt) may be used.

“Primer pair” means an oligonucleotide pair, either natural or synthetic that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

The term “nucleic acid” refers to deoxyribonucleotides or ribonucleotides and polymers thereof in either single- or double-stranded form. The term nucleic acid is used interchangeably with gene, cDNA, and mRNA encoded by a gene.

### EXEMPLARY ASPECTS

Below are examples of specific aspects for carrying out the present invention. The examples are offered for illustrative purposes only, and are not intended to limit the scope of the present invention in any way. Efforts have been made to ensure accuracy with respect to numbers used (e.g., amounts, temperatures, and the like), but some experimental error and deviation should, of course, be allowed for.

### Materials and Methods

Reference E. coli Strains.

The primary (reference) study strain collection included 191 commensal and pathogenic isolates of E. coli, including 70 strains from the E. coli reference (ECOR) collection and 38 strains with publicly available genome sequences. Two of the ECOR specimens—ECOR 43 and ECOR 59—were excluded from further study after confirmatory molecular testing failed to yield the expected profiles.

An additional 83 mainly extra-intestinal isolates were included, of which 75 have been described previously. The 8 previously unpublished strains were urine or fecal isolates from humans and domesticated animals with acute UTI and included fecal isolate HFP004, from a collection of fecal isolates recovered from women with acute UTI (and their household members) treated at a family practice clinic in the Minneapolis, Minn. area; clinical isolates JRF12A, JRF15A, JRF16A, JRF22A, JRF26A, and JRF173, from dogs or cats with acute UTI evaluated at an ambulatory veterinary practice in San Diego County, Calif.; and HI#2, a urine isolate collected from a healthy adult female with cystitis at the University of Washington, Seattle, Wash. Previously unpublished isolates were assigned to 1 of the 4 traditionally recognized E. coli phylogenetic groups (A, B1, B2, and D) based on either PCR-based phylotyping, as previously described, or clustering with reference strains in a dendrogram based on concatenated MLST sequences.

### Fresh Clinical (Current) E. Coli Isolates.

The collection of fresh clinical (current) E. coli isolates consisted of 853 consecutive E. coli isolates recovered in the clinical microbiology laboratories of several medical institutions during the routine processing of various clinical specimens-mainly urine (91%), but also wound (3%), blood (2%), and other specimens—between October 2010 and January 2011. Of the current isolates, 300 were obtained from the Group Health Cooperative (Seattle, Wash.), 200 were from the University of Washington Medical Center (Seattle, Wash.), 143 were from the Harborview Medical Center (Seattle, Wash.), 110 were from the Minneapolis Veterans Administration Medical Center (Minneapolis, Minn.), and 100 were from Seattle Children's Hospital (Seattle, Wash.).

MLST and fimH Sequencing.

Amplification and sequencing of the MLST loci were done as previously described. For fimH amplification and sequencing, the following fimH primers were used: fimH-F (SEQ ID NO: 01), CACTCAGGGAACCATTCAGGCA (binds 50 to 72 nucleotides [nt] upstream of fimH start); fimH-R (SEQ ID NO: 02), CTTATTGATAAACAAAAGTCAC (spans the last 21 nt of fimH). When necessary, the following mid primer was used to complete full-length fimH sequencing: fimH-mid (SEQ ID NO: 03), CGTTGTTTATAATTCGAG (binds nt 339 to 356 of fimH). The thermocycler program for all reactions consisted of 1 cycle of 94° C. for 5 min, followed by 30 cycles of 94° C. for 30 s, 57° C. for 15 s, and 72° C. for 1 min. Contigs were assembled using BioNumerics (Applied Maths, Sint-Martens-Latem, Belgium). To describe the predicted FimH peptides associated with each allele, the consensus FimH protein that carries the most conserved residue at each amino acid position in the mature peptide was selected as the reference. Amino acids in the signal peptide were numbered 1 (start codon) through 21 and are indicated throughout this report with a preceding minus sign.

Identification of fimH-null Strains.

Strains with publicly available genomes were assigned fimH-null status if they were found to have any interruption (e.g., by insertion sequence) or deletion of the region flanked by fimH primer annealing sites (as for 7 strains). Strains sequenced de novo for this study were assigned fimH-null status if they did not amplify a product of the expected size (975 bp) with the fimH primers used here (as for 5 ECOR strains).

### Phylogenetic Analysis.

For each strain, the seven MLST gene fragments were concatenated into a single sequence of approximately 3,500 nucleotides. PAUP* 4.0b was used to generate maximum-likelihood DNA trees for concatenated MLST sequences and for full-length fimH sequences.

Phylogenetic Analysis of Current E. coli.

Of 853 isolates, 611 (72%) had all 7 MLST genes sequenced, 5% had 6, 4.7% had 5, 6% had 4, 10% had 3, and 2% had only 2 MLST genes sequenced. The isolates that underwent less-than-full MLST analysis had a unique combination of sequenced genes that placed them in one of several major ST complexes with high probability (P<0.0001).

### Nucleotide Polymorphism Analysis.

Nucleotide polymorphism was measured by average pairwise diversity index, n, using MEGA version 4. The polymorphism plot was derived from a series of n values across overlapping windows of 100 nucleotides with a step size of 50 nucleotides using ProSeq v2.91.

### Discriminatory Power and Cluster Correlation Analyses.

Discriminatory power was analyzed using Simpson's index of diversity (D) (12), which quantifies the likelihood that two individuals selected randomly from the same population will exhibit different types. Thus, the relative discriminatory power of two typing methods can be compared directly using D when they are applied to the same population. Correlation of clustering techniques was evaluated using the Wallace coefficient, which measures the probability that paired strains assigned to the same genotype group by one method are also classified in the same type by the other method. The publicly available script described by Carrico and colleagues on the World Wide Web at biomath.itqb.unl.pt/ClusterComp was implemented in BioNumerics.

### Isolates and Patients.

The primary set of 1,518 recent clinical E. coli isolates consisted of consecutive single-patient human extraintestinal isolates recovered between October 2010 and June 2011 at five clinical microbiology laboratories serving distinct patient populations in Seattle, Wash. (Group Health Cooperative, Harborview Medical Center, Seattle Children's Hospital, and the University of Washington Medical Center), and Minneapolis, Minn. (Veterans Affairs Medical Center). For 20% of the isolates (those from Group Health), the supplying clinical laboratory reported that the isolates came almost exclusively from urine specimens, which agrees with the clinical context in that Group Health serves ambulatory patients only. For the rest of the collection, 93% of those isolates were from urine, 2% from blood, and 5% from miscellaneous other sample sites (sputum, wound, abscess, etc.). Another set of E. coli isolates was obtained from the University Hospital in Munster, Germany, and consisted of 161 consecutive isolates from urine samples recovered from July to September 2012.

Among the 1,518 primary set isolates, data regarding the presence of sepsis were available for 1,133 isolates, and data regarding the persistence or recurrence of infection were available for 1,034 urine isolates. The latter were classified as (i) single-episode bacteriuria (no clinical or microbiological evidence of recurrence within 30 days after the index culture) or (ii) recurrent UTI (clinical or microbiological evidence of persistence or recurrence beyond 7 days and within 30 days following the initial resolution of symptoms). Drug-bug mismatch in relation to the initially chosen antimicrobial therapy was analyzed in 676 urine isolates within the primary set (for 10 out of 676 patients, information about recurrence or persistence of infection was not available). The specific regimens used were diverse, and because of small subgroups, a detailed analysis of each regimen in relation to an organism would provide too little value to justify its inclusion in the study. The most relevant consideration analyzed and summarized in the report is whether the particular regimen chosen was active in vitro against each patient's infecting organism. Local institutional review boards approved the study protocol.

### Susceptibility Testing.

Antimicrobial susceptibility profiles were determined using disk diffusion testing. The interpretive criteria were those specified by the Clinical and Laboratory Standards Institute (CLSI).

Clonal Typing of E. coli Isolates.

Internal (<500 bp) regions of fumC and fimH were amplified by PCR, and the DNA sequences were determined by Sanger sequencing. Each unique combination of fumC and fimH alleles defined a CH clonotype. The diversity of the clonotype distribution was evaluated using the Simpson's modified alpha-diversity index.

### Statistical Analysis of Antimicrobial Susceptibility of Major CH Clonotypes.

The prevalence of susceptibility to individual agents was calculated for the total primary collection, for individual participating laboratories, and for individual CH clonotypes. Odds ratios (ORs) were calculated for each subgroup (i.e., laboratory or clonotype) relative to the rest of the population. If the OR was >2 or <0.5, its statistical significance was evaluated using Fisher's exact test.

### Clonotype-Guided Algorithm for Prediction of Antimicrobial Resistance.

Each isolate in the primary collection was classified as resistant or susceptible to each of the 11 antimicrobials based on the prevalence of susceptibility to the particular antimicrobial in the rest of isolates belonging to the same clonotype (i.e., for each isolate classification, the susceptibility of the corresponding clonotype was recalculated by excluding the profile of the isolate to be classified). Thus, clonotype-guided antibiotic selection was evaluated only for isolates from clonotypes comprising greater than or equal to 2 isolates. An isolate was hypothetically “allowed” to be treated with that agent if its clonotype susceptibility to that agent was greater than or equal to 80%, whereas if its susceptibility was <80%, it was classified as resistant and was “rejected” for treatment with the agent.

### Clonotype Identification Directly in Urine Specimens.

Greater than 50 clinical urine specimens (generally, using the boric acid preservative that allows urine to be kept at room temperature) were submitted to microbiology laboratories for the culture and susceptibility tests. One milliliter of each urine specimen was spun down for 1 minute at 10,000 rpm, the sediment was resuspended in 100 microliters of distilled water, and heated at 98° C. for 5 min. This sample was used at a final dilution of 1:10 for either quantitative PCR (qPCR) or pyrosequencing testing. qPCR was performed on LightCycler 2.0 (Hoffmann-La Roche, Inc.) using gene- or single-nucleotide polymorphism (SNP)-based primers specific to the clonotype CH40-30. The gene-based probes targeted the rMST1 gene region described earlier (7). Pyrosequencing was performed on PyroMark Q24 (Qiagen, Inc.), targeting the short most-clonotype-variable (<60 bp) internal regions of fimH and fumC.

### Example 1

### Discriminatory Power and Congruence of fimH and MLST

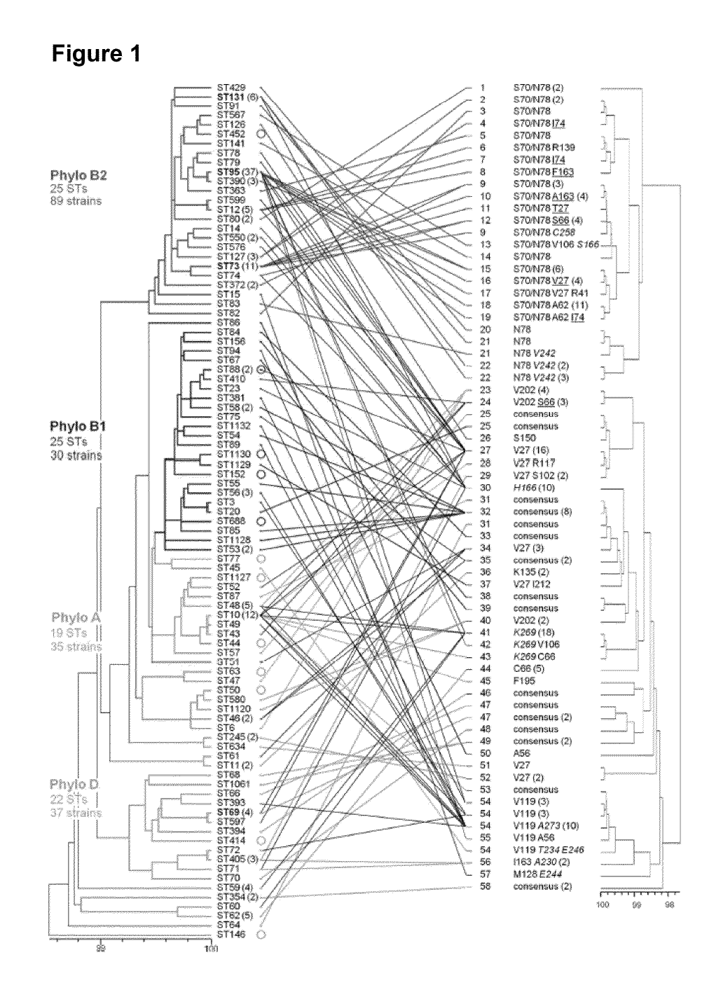

To evaluate the suitability of fimH as a typing locus, the 7-locus MLST profiles and full-length fimH sequence of 191 reference E. coli strains were determined. The individual MLST loci exhibited 26 to 37 alleles each (Table 1). An intact, full-length fimH sequence was obtained from 179 (94%) of the 191 reference strains, with 67 unique, full-length fimH alleles observed; the 12 fimH-nullπ strains derived from the ECOR (5 strains) and publicly available genome (7 strains) collections. Thus, fimH exhibited greater sequence variation than the individual MLST housekeeping genes. The congruence of the fimH and MLST phylogenies was examined next. In total, 91 unique MLST profiles (STs) were encountered among the reference strains, all differing by at least 1 nt in one locus and spanning the 4 traditionally recognized phylogenetic groups of E. coli, i.e., groups A, B1, B2, and D (FIG. 1, left panel). Of the 67 full-length fimH alleles, 58 were associated with a single phylogenetic group. This subset included the 25 alleles encoding FimH polymorphism N78 (FIG. 1, Phylo B2), all of which were associated with phylogenetic group B2, and 7 alleles that appeared in multiple STs but all within a given phylogenetic group (FIG. 1; Phylo B1, Phylo A and Phylo D). Thus, these fimH alleles could be defined as phylogenetically restricted alleles. The remaining 9 alleles were associated with STs in 2 or more phylogenetic groups (FIG. 1, black cross lines), indicating that certain fimH alleles frequently move horizontally among phylogenetically distant lineages of E. coli and could be defined as phylogenetically dispersed alleles.

TABLE 1
Numbers of types found and D values of individual
and combined loci of 191 diverse _{E. coli} isolates

| | | | |
|---|---|---|---|
| Typing method | # of types found | D | 95% CI |

Single loci

| | | | |
|---|---|---|---|
| adk | 35 | 0.890 | (0.869-0.919) |
| fumC | 37 | 0.911 | (0.887-0.935) |
| gyrB | 37 | 0.887 | (0.858-0.915) |
| icd | 31 | 0.888 | (0.865-0.915) |
| mdh | 26 | 0.851 | (0.810-0.891) |
| purA | 28 | 0.839 | (0.802-0.877) |
| recA | 29 | 0.893 | (0.869-0.912) |
| fimH | 68 | 0.967 | (0.959-0.976) |
| fimHTR | 59 | 0.962 | (0.953-0.972) |

Loci paired with fimH

| | | | |
|---|---|---|---|
| adk + fimHTR | 99 | 0.986 | (0.982-0.991) |
| fumC + fimHTR | 102 | 0.988 | (0.983-0.992) |
| gyrB + fimHTR | 103 | 0.987 | (0.983-0.992) |
| icd + fimHTR | 98 | 0.987 | (0.983-0.992) |
| mdh + fimHTR | 96 | 0.986 | (0.981-0.990) |
| purA + fimHTR | 95 | 0.986 | (0.982-0.990) |
| recA + fimHTR | 98 | 0.987 | (0.983-0.991) |
| MLST alone | 91 | 0.951 | (0.930-0.972) |
| MLST + fimH | 126 | 0.991 | (0.986-0.995) |
| MLST + fimHTR | 123 | 0.990 | (0.986-0.995) |

### Example 2

### Trimming fimH for Typing Applications

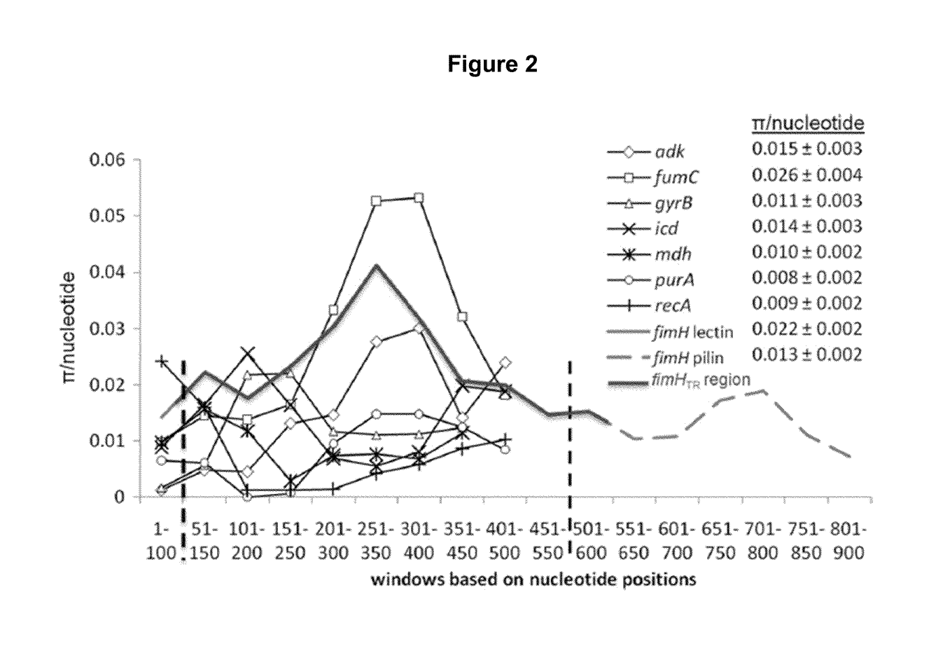

Human Sequence typing customarily uses a relatively short region of each locus (400 to 500 bp) to allow sequence determination by using only two primers. To identify an internal fragment of fimH suitable for typing purposes, sequence polymorphism analysis was performed on the 67 unique full-length fimH sequences in the reference collection. The distribution of polymorphisms was measured between the two functional domains of fimH: the N-terminal lectin domain (encoded by nt 64 to 540), which contains the mannose-specific binding pocket, and the C-terminal pilin domain (nt 541 to 900), which anchors the FimH subunit to the type 1 fimbrial shaft (pilus). According to π values (average number of polymorphisms per nucleotide), the lectin domain (overall n=0.022) was significantly more diverse (P_{—}0.02) than the pilin domain (overall π=0.013; FIG. 2). The lectin domain-encoding region of fimH was actually more diverse than 6 of the 7 MLST loci (π range of 0.008 to 0.015, P<0.05) and comparable only to that of fumC (π=0.026; P=0.37).

A location of “hot-spot” amino acid residues within FimH was considered. This region has repeatedly been targeted by amino acid replacement mutations that have pathogenicity-enhancing (pathoadaptive) effects on E. coli (36). In the fimH sequences of the reference strains, a total of 4 hotspots were identified, 3 of which (codons 27, 66, and 74) occurred within the lectin domain of FimH and the fourth of which (codon 163) occurred within the proximal portion of the pilin domain.

Using these data, a 489-bp segment was identified (here referred to as the fimH typing region [fimHTR]) that begins at the first codon of the mature peptide and ends after mature peptide codon 163 (nt 550 to 552; FIG. 2, approximated in red).

### Example 3

### Discriminatory Power of fimHTR-Based Typing

Within the reference collection, fimHTR distinguished 58 alleles, in comparison to the 67 alleles distinguished by full-length fimH. For typing purposes, fimH-null status was also defined as an additional character state (i.e., as an additional “allele”). According to the Simpson's D diversity index estimates, although full-length fimH distinguished more alleles than fimHTR, the discriminatory powers (i.e., the population diversity based on the locus sequence) of these 2 regions were nearly equivalent, with D 0.967 (confidence interval [CI], 0.959 to 0.976) for full-length fimH and D_{—}0.962 (CI, 0.953 to 0.972) for fimHTR. Thus, each exceeded the discriminatory power of individual MLST loci and was not different from that of 7-locus MLST (Table 1).

Each of the 7 MLST loci was evaluated to select the best candidate for pairing with fimHTR to increase typing resolution. Among the 7 MLST loci, fumC demonstrated numerically the greatest discriminatory power (D=0.911; CI, 0.887 to 0.935), although the values overlapped with most remaining loci (Table 1). Pairing fimHTR with fumC produced the numerically highest discriminatory power (D=0.988; CI, 0.983 to 0.992) of all such pairings and significantly exceeded the discriminatory power of full MLST (Table 1), although again the discriminatory power of the fimHTR-fumC pairing was not significantly different from that of the other pairings.

However, another attractive feature of fumC that recommended it for pairing with fimHTR in the typing scheme is the fact that, of the 7 MLST loci, fumC demonstrated the best congruence with both ST profiles and major phylogenetic groups (Table 2). These relationships were measured by the Wallace index, which expresses the probability that paired strains assigned to the same genotype group by one method are also classified in the same type by the other method. The superior phylogenetic congruence of fumC is particularly important considering the congruence disrupting effect of the phylogenetically dispersed fimH alleles, as discussed above. Therefore, the fumC fimHTR combination were selected as the target loci for sequence typing and was designated as the CH (fumC fimH) typing scheme.

TABLE 2
Correspondence of individual MLST loci with full
ST profiles and phylogenetic groups of 191 diverse
E. coli isolates using the Wallace index.

| Locus | Wallace index for ST | Wallace index for phylogenetic group |
|---|---|---|
| adk | 0.462 | 0.800 |
| fumC | 0.548 | 0.986 |
| gyrB | 0.432 | 0.900 |
| icd | 0.437 | 0.944 |
| mdh | 0.328 | 0.959 |
| purA | 0.305 | 0.766 |
| recA | 0.459 | 0.892 |
| fimH_{TR} | 0.258 | 0.504 |

### Example 4

### Correlation Between MLST and CH Typing Among Current E. coli Isolates

To determine the resolution and specificity of CH typing in a field application, 853 fresh clinical E. coli isolates were analyzed. The isolates were collected consecutively as part of routine diagnostics in five different clinical microbiology labs, from October 2010 through January 2011, without any pre-selection criteria. All isolates were of extra-intestinal origin, primarily from urine. The MLST loci could be sequenced in all of the isolates tested, while fimHTR could be sequenced in more than 99% of the isolates (n=846).

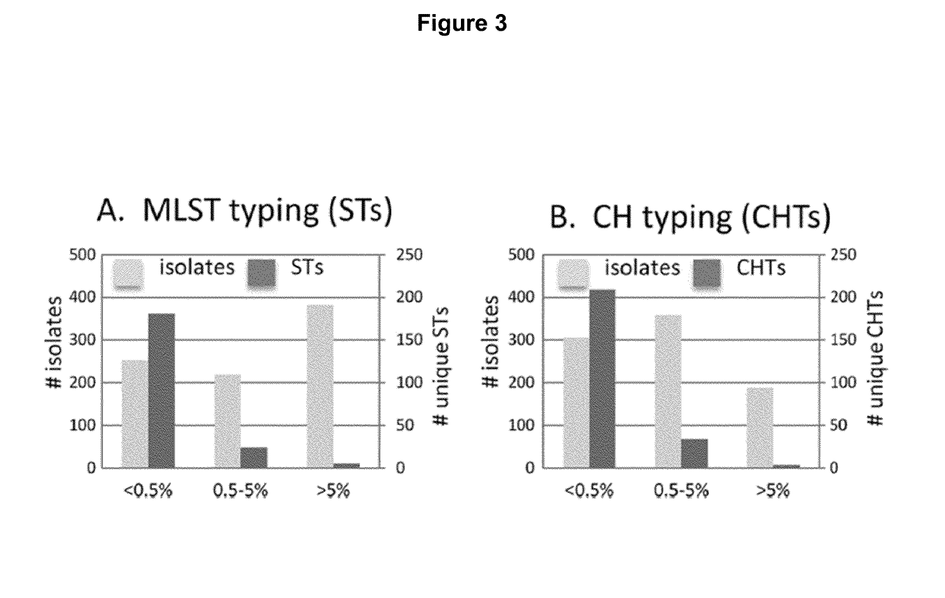

In total, 210 unique MLST profiles (i.e., STs) were identified. Among them, 181 small STs each comprised <0.5% of the population (≦4 isolates; FIG. 3A), collectively accounting for 252 isolates (29.5%). Additionally, 24 medium STs each comprised 0.5 to 5% of the population (5 to 35 isolates in the collection), collectively accounting for 219 isolates (25.7%). Finally, 5 large STs each included more than 5% of the population (≦40 isolates in the collection) each, collectively accounting for 382 isolates (44.9%). Thus, while the number of individual STs decreased progressively along a gradient from small to large ST size, the greatest proportion of current isolates was accounted for by relatively few large STs, evidence of the highly clonal structure of clinical ExPEC isolates.

The current clinical isolates carried 143 unique fimHTR alleles. When fumC and fimHTR were combined for CH typing, there were a total of 246 unique CH types (CHTs), i.e., more than the number of 7-locus STs (see above). Similar to STs, the number of CHTs decreased significantly from small (209 CHTs) to medium (34 CHTs) to large (3 CHTs) (FIG. 3B). However, compared to STs, the absolute number of small and medium CHTs was somewhat greater, while the number of large CHTs was significantly lower. Likewise, whereas with MLST the aggregated large STs were most numerous, with CH typing the aggregated medium CHTs were most numerous, indicating that CH typing splits larger STs into smaller CHTs.

To determine to what extent unique CHTs correspond with MLST-based clonal groups, STs were combined into ST complexes by using the eBURST program (on the World Wide Web at eburst.mlst.net), where each ST must match at 6 of 7 loci with at least 1 other ST in the complex. Nearly half of the singleton STs (66 of 138) could be combined this way with another ST within the collection, with the rest remaining as individual STs; this yielded a total of 123 separate STs or ST complexes. Overall, 224 CHTs (i.e., >90% of the total) had a unique, specific match and another 3 CHTs were mostly (93 to 97%) matched to a single ST or ST complex. This gave an overall match rate between CH typing and MLST of 95.8%.

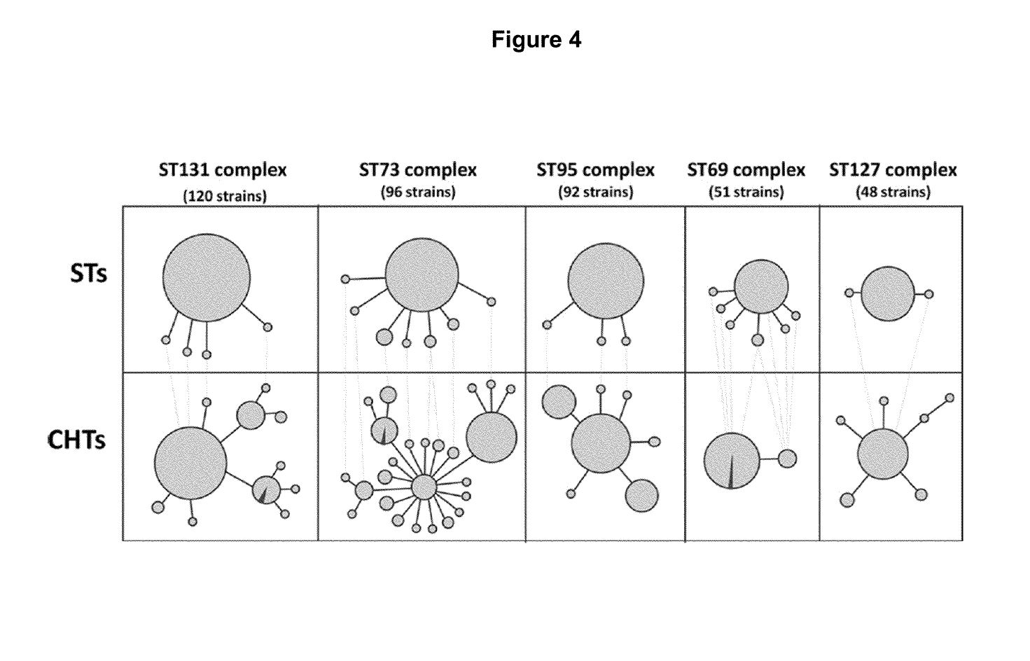

The overall superior resolution and the clonal matching of CH typing relative to MLST are illustrated in FIG. 4, where the 5 largest ST complexes (each represented by >5% of the isolates) are compared directly to the corresponding CHTs. These large ST complexes included such notorious ExPEC clones as ST131, ST73, ST95, ST69, and ST127 (FIG. 4, upper panel). Only in the ST69 complex was the number of CHTs less than the number of STs within the same complex. In the other 4 ST complexes, CHTs outnumbered the corresponding STs by 2- to 3-fold. Furthermore, within each complex, except the ST69 complex, the major (founder) ST was split into 4 to 15 different CHTs. For the large ST complexes, almost all CHTs were specific to that complex (FIG. 4), with an overall match rate of 98.8%.

Thus, among current ExPEC isolates, 2-locus CH typing provided discriminatory power superior to that of MLST while maintaining robust clonal correspondence with the MLST-based clonal groups.

The data above demonstrate a two-locus, sequence-based typing scheme for Escherichia coli that utilizes a 489-nucleotide internal fragment of fimH (encoding the type 1 fimbrial adhesin) and the 469-nt internal fumC fragment used in standard MLST. Based on sequence typing of 191 model commensal and pathogenic isolates plus 853 freshly isolated clinical E. coli strains, this 2-locus approach (termed here CH typing (fumC/fimH)) consistently yielded more haplotypes than standard 7-locus MLST, splitting large STs into multiple clonal subgroups and often distinguishing different within-ST eco- and pathotypes. Furthermore, specific CH profiles corresponded to specific STs, or ST complexes, with 95% accuracy, allowing excellent prediction of MLST-based profiles

### Example 5

### Major Clonotypes Dominate within E. coli Across Different Clinical Laboratories

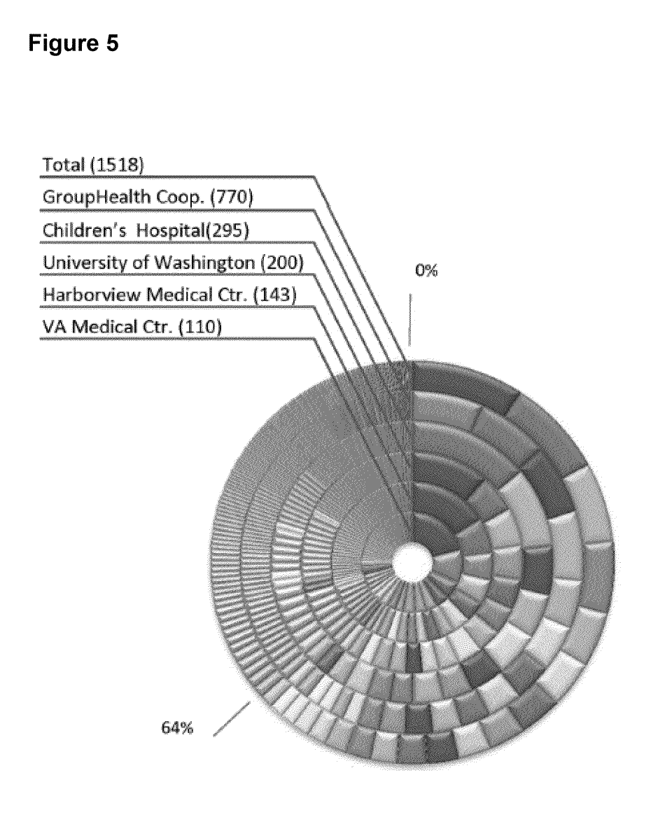

A total of 222 distinct CH clonotypes were identified among 1,518 U.S.-based clinical E. coli isolates, comprising 1 to 137 isolates each (FIG. 5). Clonotypes consisting of a single isolate comprised only 7% of all isolates. Nineteen clonotypes that consisted of greater than or equal 15 isolates (i.e., greater than or equal to 1% of the collection) were defined as major (FIG. 5). Clonotype distribution was highly similar across laboratories (FIG. 5), with at least three of the four largest clonotypes overall predominating within each laboratory. The largest clonotype, both overall and at three of the five contributing laboratories, was CH40-30 from sequence type 131 (ST131).

Overall, 96 clonotypes were encountered in isolates from at least two different laboratories. In total, the common clonotypes comprised 89.6% of isolates, demonstrating a highly clonal structure of the vast majority of extraintestinal E. coli isolates, found across different geographic areas and patient populations.

### Example 6

### Antimicrobial Susceptibility Profiles of Clonotypes are Distinct and Consistent Across Different Locations

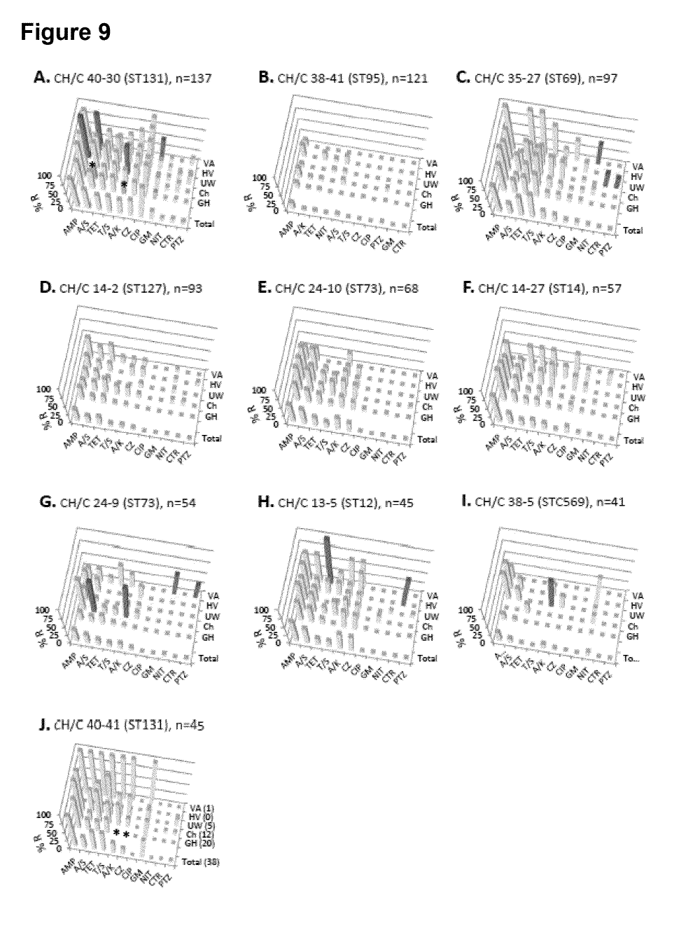

Within each of the 19 major clonotypes, the prevalence of resistance differed by greater than or equal to 2-fold (higher or lower) (P<0.05) from that of the rest of population for at least one antimicrobial (FIG. 6). Resistance prevalence within a clonotype did not correlate with the overall population prevalence of the clonotype; for example, among the most-prevalent clonotypes were several of the extensively resistant (e.g., CH40-30 and CH35-27) and the extensively susceptible (e.g., CH38-41 and CH14-2) clonotypes. For a given major clonotype, resistance patterns were highly consistent across laboratories, with only a few statistically significant interlaboratory differences (see FIG. 9 A to G).

### Example 7

### E. coli Clonal Typing Improves Prediction of Isolate Susceptibility to Antibiotics

Based on the greater than or equal to 80% susceptibility cutoff triage, CH-based typing allowed for treatment with a particular agent for widely divergent proportions of the isolates (Table 3). Among 5 oral antimicrobials that were most commonly used to treat E. coli infections in the data set, amoxicillin-clavulanate was allowed for 48.1% of isolates, cefazolin for 58%, trimethoprim-sulfamethoxazole for 58%, fluoroquinolones for 79.4%, and nitrofurantoin for 94.1%. In the allowed population, the actual prevalence of resistance to the corresponding antimicrobials ranged from 21.9% (ampicillin) to 3.74% (fluoroquinolones). The relative potential improvement in the prediction of susceptibility to a given agent with the clonotyping based approach, compared with the total observed susceptibility, ranged from 45.4% (cefazolin) to 78.1% (fluoroquinolones), with an average improvement of 57.4%.

TABLE 3
Performance statistics for clonotype-based susceptibility predictions
for the primary collection of clinical E. coli isolates^{a}

| | | Performance of clonotype-based choice | | |
|---|---|---|---|---|
| | Observed | of antimicrobial agent (%)^{c} | | |
| | Resistance rate | Rejected/ | Allowed/ | |
| Antibiotic^{b} | (%)^{d} | Resistance | Resistance | Improvement^{e} |
| AMP^{f} | 51.6 | 77.7/60.1 | 22.3/21.9 | 57.5 |
| TET^{f} | 29.5 | 49.4/48.0 | 50.6/11.5 | 61.1 |
| A/S^{f} | 29.4 | 66.2/37.9 | 33.8/13.0 | 55.9 |
| T/S | 26.9 | 42.0/50.1 | 58.0/10.1 | 62.4 |
| A/K | 25.5 | 51.9/36.5 | 48.1/13.5 | 46.8 |
| CZ | 19.7 | 42.0/32.0 | 58.0/10.7 | 45.4 |
| CIP | 17.1 | 20.6/68.7 | 79.4/3.7 | 78.1 |
| GM^{f} | 8.92 | 17.1/31.4 | 82.9/4.3 | 52.1 |
| NIT | 6.79 | 5.9/27.4 | 94.1/5.5 | 19.2 |
| CTR | 5.38 | 4.3/31.1 | 95.7/4.2 | 21.6 |
| PTZ | 3.96 | 2.1/13.3 | 97.9/3.8 | 5.1 |

^{a}A total of 1,518 isolates were typed using a fumC-fimH (CH) scheme and were tested against 11 antimicrobials. Of the isolates, 1,413 out of 1,518 belonged to CH clonotypes that contained >1 isolate (nonsingletons).
^{b}AMP, ampicillin; TET, tetracycline; A/S, ampicillin-sulbactam; T/S,
trimethoprimsulfamethoxazole; A/K, amoxicillin-clavulanate; CZ, cefazolin; CIP, ciprofloxacin; GM, gentamicin; NIT, nitrofurantoin; CTR, ceftriaxone; PTZ, piperacillin-tazobactam.
^{c}For each isolate, the treatment with an antimicrobial agent was allowed or not based on the prevalence of the susceptibility to this agent in the respective CH clonotype; to avoid bias, each analyzed isolate was excluded from the calculation of prevalence.
^{d}Rate (%) of resistant isolates among 1,413 isolates.
^{e}Percent improvement toward ideal test (100%) was calculated as (difference between the CH and antibiogram approach)/(difference between the antibiogram approach and 100%) × 100; all improvement rates were statistically significant (P < 0.001, Fisher's exact test), except for with NIT and CTR (P = 0.09) and PTZ (P = 0.43).
^{f}Antimicrobials with comparatively limited clinical utility.

### Example 8

### Clonotyping Predicts Antimicrobial Susceptibilities Across Different Locations

The clonotype-guided susceptibility prediction analyses for ciprofloxacin (a fluoroquinolone), which was prescribed in 40% of patients, and trimethoprim-sulfamethoxazole were done separately for the isolates from each clinical laboratory (Table 4), including for a set of 161 E. coli urine isolates from the University Clinics Hospital in Munster, Germany. To avoid potential self-selection bias in the analysis of these smaller groups, the susceptibility classification for the isolates in each subset was done after excluding the susceptibility profiles of that particular subset, i.e., by using the clonotype susceptibility profiles of the remaining isolates from common clonotypes (Table 4).

TABLE 4
Performance of clonotype-based susceptibility predictions compared to observed
susceptibility among clinical E. coli isolates from different laboratories

| | Clinical microbiology laboratory and associated performance (%)^{a} | | | | | |
|---|---|---|---|---|---|---|
| | Group | | | Harborview | | |
| Resistance and | Health | Children's | Univ. of | Med. | VA Med. | Univ. of |
| improvement rates | Co-op, | Hospital, | Washington, | Center, | Center, | Münster, |
| by antibiotic type^{b} | Seattle | Seattle | Seattle | Seattle | Minneapolis | Münster |

T/S

| | | | | | | |
|---|---|---|---|---|---|---|
| Total Resistant | 20.1 | 34.2 | 33.3 | 30.4 | 34.0 | 32.6 |
| Rejected/resistance | 48.8/36.2 | 41.9/58.7 | 43.5/62.6 | 45.6/58.3 | 50.0/51.9 | 56.6/46.6 |
| Allowed/resistance | 51.2/5.0 | 58.1/14.2 | 56.5/11.4 | 54.4/7.4 | 50.0/17.0 | 43.4/14.3 |
| Improvement^{c} | 72.9 | 49.7 | 65.7 | 75.8 | 50.0 | 56.1 |

CIP

| | | | | | | |
|---|---|---|---|---|---|---|
| Total Resistant | 13.7 | 10.0 | 21.5 | 31.2 | 35.1 | 30.2 |
| Rejected/resistance | 17.5/59.6 | 16.5/54.5 | 26.3/71.2 | 32.8/88.6 | 39.4/72.9 | 33.3/74.4 |
| Allowed/resistance | 82.5/4.1 | 83.5/1.4 | 73.7/3.6 | 67.2/3.6 | 60.6/7.0 | 66.7/8.14 |
| Improvement^{c} | 70.1 | 86.2 | 83.0 | 88.6 | 80.0 | 73.1 |

^{a}The set of isolates for which susceptibility was predicted was based on the mean susceptibility of the clonotype to which they belong. This mean susceptibility was calculated in each case for all isolates minus the isolates belonging to the validation set.
^{b}T/S, trimethoprim-sulfamethoxazole; CIP, ciprofloxacin.
^{c}Percent improvement was calculated as difference between resistance (i.e., potential drug-bug mismatch) in CH-allowed cases and observed resistance based on the actual susceptibility data (divided by the latter, ×100%); all improvements were statistically significant (P < 0.001, Fisher's exact test) unless stated otherwise.

Based on the greater than or equal to 80% susceptibility cutoff, CH-based typing rejected treatment with trimethoprim-sulfamethoxazole in 41.9 to 56.6% (mean, 47.7%) of the isolates, depending on the laboratory. Among the remaining allowed isolates, the actual prevalences of trimethoprim-sulfamethoxazole resistance were between 5.0% and 17.2% (mean, 12.1%), which was 2- to 4-fold lower than the overall resistance in the corresponding site. For ciprofloxacin, CH-based typing rejected treatment in 16.5 to 39.4% (mean, 27.6%) of the isolates. In the remaining allowed population, the actual prevalences of resistance to ciprofloxacin ranged from 8.1% to 1.4% (mean, 4.6%), which was 3- to 9-fold lower than the overall resistance prevalence.

### Example 9

### Association of UTI Persistence or Recurrence with Clonotypes and Drug-Bug Mismatch

For 1,034 urine isolates, follow-up patient data were available (see Table 5). More than 13% (n=135) of these patients experienced a persistent or recurrent UTI within 30 days. The overall clonal diversity of the isolates associated with persistent or recurrent UTI was significantly lower than that of the remaining isolates (P<0.001) (see Table 6). Thus, a relatively limited subset of CH-based clonotypes of E. coli has an enhanced propensity to cause persistent or recurrent UTIs. Indeed, one clonotype, CH40-30, predominated and was significantly overrepresented among patients with persistent or recurrent UTIs (FIG. 7) (OR, 3.7; P<0.001). Conversely, certain other clonotypes, including CH38-41 and CH13-5 (P<0.05), and possibly CH38-15 and CH24-10 (P<0.10), were negatively associated with persistence or recurrence of infection (FIG. 7).

The possible effects of drug-bug mismatch on persistence or recurrence of infection was assessed next. For this, actual clinical practices were analyzed for the subset of urine isolates (n=666) that were documented to have been treated empirically with only one antimicrobial agent prescribed at or around the day of the visit. The empirically prescribed agents (among the 666 patients with available data) were fluoroquinolones (267 [38%]), trimethoprim-sulfamethoxazole (TMP-SMX) (181 [25%]), first-generation cephalosporins (83 [11%]), third-generation cephalosporins (73 [10%]), nitrofurantoin (45 [6.4%]), penicillins (16 [2.3%]), and second-generation cephalosporins (11 [1.5%]), plus carbapenems, amoxicillin-clavulanate, ampicillin-sulbactam, tetracycline, and gentamicin (each <1%). Of these isolates, 99 (15%) were resistant to the empirically prescribed antimicrobial agent and 27% (27/99) of them were present in persistent or recurrent UTIs (FIG. 7) versus only 10.6% (60/567) of those whose isolate was susceptible to the initially prescribed agent (P<0.001).

TABLE 5
Description of the primary set of clinical E. coli isolates (2010-2011)

| | | | Median Patient | | # of isolates for analysis of: | | |
|---|---|---|---|---|---|---|---|
| | | | Age, yrs | Unique CH | Recurrence/ | | Antimicrobial |
| Center | Location | Isolates | (min, max) | clonotypes | Persitance | Sepsis | Therapy |
| Children's | Seattle, WA | 294 | 5 (3 d, 22) | 75 | 246 | 269 | 169 |
| Hospital | | | | | | | |
| University of | Seattle, WA | 200 | 49 (18, 94) | 70 | 154 | 158 | 103 |
| Washington | | | | | | | |
| Harborview | Seattle, WA | 143 | 58 (18, 92) | 63 | 111 | 135 | 48 |
| Med. Ctr. | | | | | | | |
| Group | Seattle, WA | 771 | 58 (18, 98) | 151 | 441 | 471 | 309 |
| Health Co-op | | | | | | | |
| VA Med. | Minneapolis | 110 | 68 (21, 98) | 46 | 82 | 100 | 47 |
| Ctr. | MN | | | | | | |
| TOTAL | | 1518 | 48 (3 d, 98) | 220 | 1034 | 1133 | 676 |

a Except when stated otherwise (i.e., d—days)
b Note that the data on recurrence/persistence for this set were available only for 666 isolates

TABLE 6
Clonotype diversity in relation to sepsis
and UTI recurrence/persistence

| | | | Diversity paramenters^{d} | |
|---|---|---|---|---|
| | # of strains | # of clonotypes | Simpson index^{b} | Alpha index^{c} |
| UTI | 135 | 54 | 0.072 ± 0.014 | 33 ± 4.6 |
| Recurrent/ | | | | |
| persistant^{a} | | | | |
| Single | 899 | 166 | 0.035 ± 0.002 | 60 ± 3.3 |
| Sepsis | 59 | 25 | 0.082 ± 0.015 | 16.4 ± 3.5 |
| No sepsis | 1074 | 188 | 0.035 ± 0.002 | 66 ± 11 |

^{a}Recurrence/persistence was analyzed for urine isolates, whereas sepsis was analyzed for all isolates
^{b}Simpson index here is the probability that any two isolates belong to the same clonotype; greater Simpson index indicates less diversity
^{c}Alpha index measures diversity based on alpha model which assumes that the abundance for each clonotype follows a Poisson distribution; lower alpha index indicates less diversity
^{d}Differences in diversity are statistically significant with P < .0001 for Alpha indexes and P = 0.012 and P = 0.002 for Simpson indexes for recurrence/persistence and sepsis, respectively

### Example 10

### Association of Clonotypes with Sepsis

Of the 1,518 primary set isolates, 1,133 were from patients with available clinical diagnostic data, of whom 59 were diagnosed with sepsis (see Table 5). The overall clonotype diversity of the 59 sepsis-associated isolates was significantly more limited than that of the 1,074 remaining isolates (P<0.001) (see Table 6), indicating that relatively few E. coli CH clonotypes are predisposed to cause bloodstream infections. Among the sepsis-associated isolates, the most prevalent clonotype was CH40-30 (17% of sepsis cases versus 8.9% of patients without sepsis; OR, 2.1) (P=0.04) (FIG. 7). Other clonotypes associated with sepsis were CH14-2 (13.3% versus 6.33%; OR, 2.3) (P=0.039) and, potentially, CH4-27 (5.1% versus 2.3%; OR, 2.3) (P=0.096) (see FIG. 7).

### Example 11

### Clonotype Identification Directly in Urine Specimens

It was determined whether clonotype information about E. coli could be determined directly in the patients' urine specimens by using common molecular diagnostics instruments, the LightCycler 2.0 (Roche Diagnostics GmbH) and PyroMark Q24 (Qiagen, Inc.), which utilize quantitative PCR (qPCR) and pyrosequencing approaches, respectively.

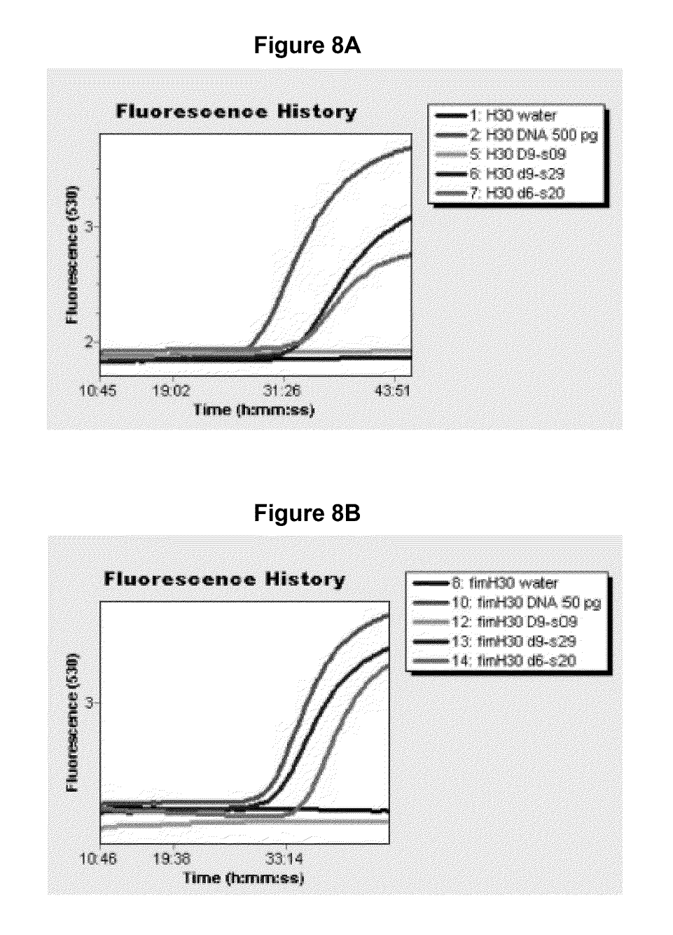

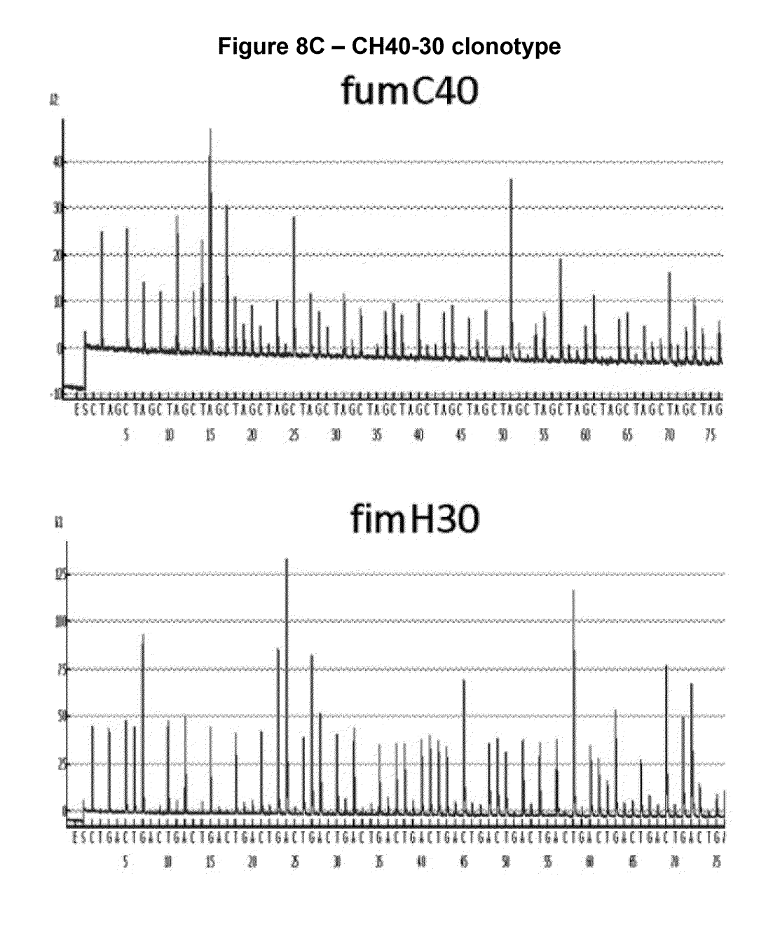

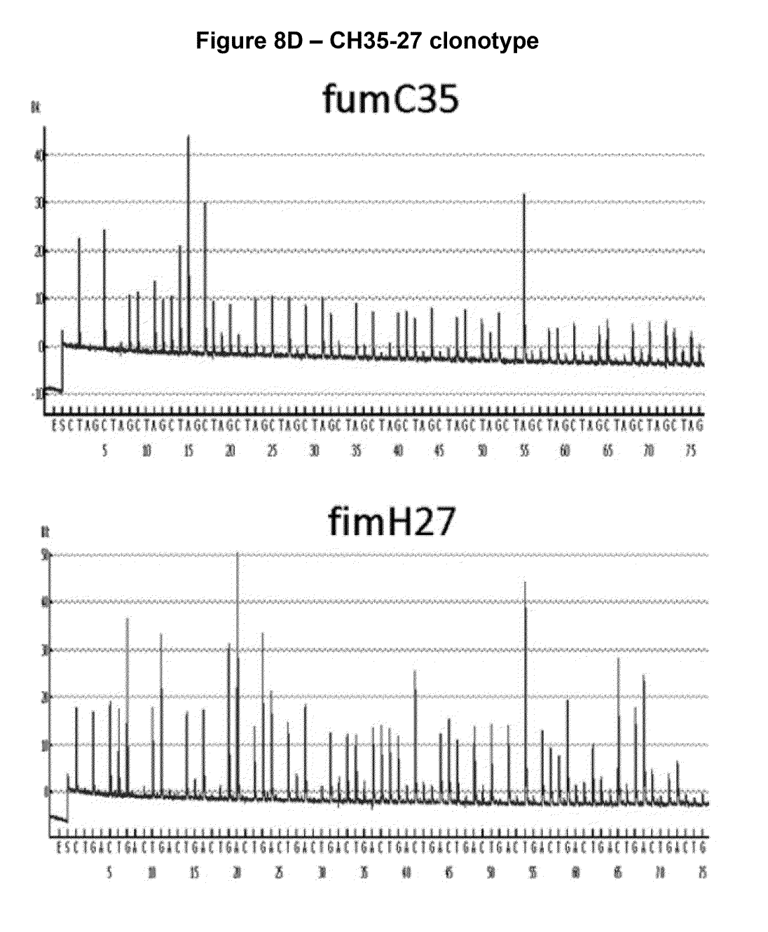

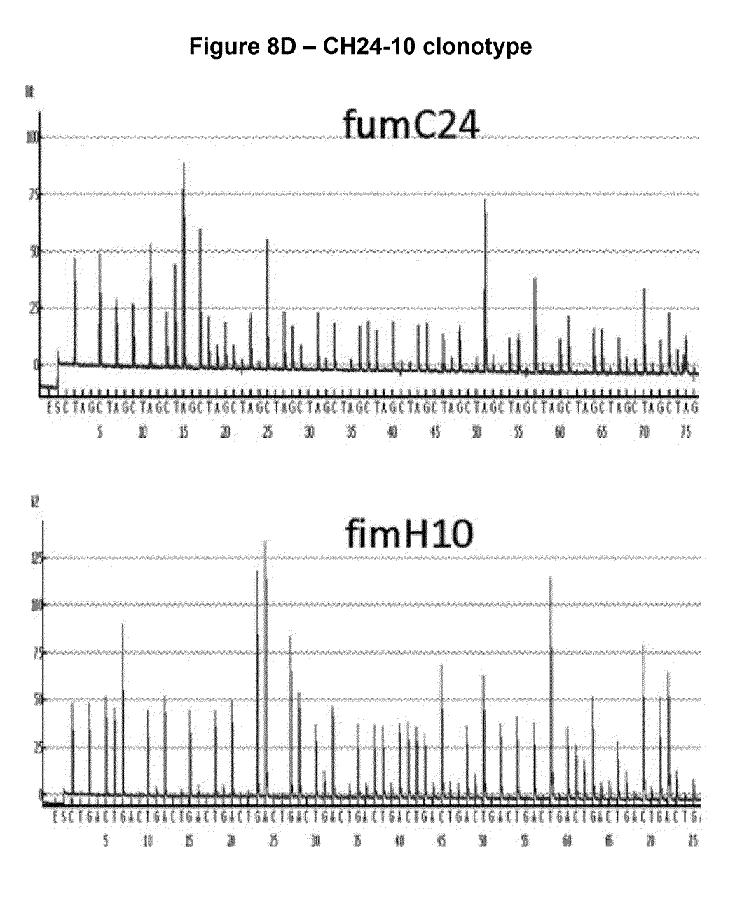

The qPCR diagnostic tests were based on gene-specific or SNP-specific probes, thus allowing for the detection of one clonotype in a single test run. Two probes capable of detecting the predominant CH40-30 clonotype were designed, one based on the rMST1 gene region and another on a canonical SNP in one of the CH clonotyping loci, fimH. The qPCR probes detected the infecting bacteria directly in the patients' urine specimens (FIGS. 8A and B) in all specimens containing the CH40-30 E. coli clonotype with a bacterial load of greater than or equal to 10⁴ per ml. By using qPCR, the clonotype identity was determined within 1 hour upon starting processing of a clinical specimen.

Unlike qPCR, pyrosequencing-based tests provide information about the nucleotide sequences of specific gene regions. Thus, this method allows for the detection of many different clonotypes in a single test run, depending on the sequence diversity of the target gene region. Test primers against short (<60 bp) highly polymorphic regions within the fumC and fimH loci were used and, as with qPCR, the clonotype identities of the bacterial isolates were determined directly in patient urine specimens. Illustrative results detecting bacteria identified as belonging to the CH40-30, CH35-27, and CH24-10 clonotypes are presented in FIGS. 8C, 8D and 8E, respectively. Different E. coli clonotypes were identified within the clinical specimens that had bacterial loads of greater than or equal to 10³ per ml. The pyrosequencing was accomplished generally within 3 hours upon the start of processing a clinical specimen.

The examples above demonstrate that splitting Escherichia coli species into clonal groups (clonotypes) predicts antimicrobial susceptibility or clinical outcome. A total of 1,679 E. coli isolates (collected from 2010 to 2012) were collected from one German and 5 U.S. clinical microbiology laboratories. Clonotype identity was determined by fumC and fimH(CH) sequencing. The associations of clonotype with antimicrobial susceptibility and clinical variables were evaluated. CH typing divided the isolates into >200 CH clonotypes, with 93% of the isolates belonging to clonotypes with >2 isolates. Antimicrobial susceptibility varied substantially among clonotypes, but was consistent across different locations. Clonotype-guided antimicrobial selection significantly reduced “drug-bug” mismatch compared to that which occurs with the use of conventional empirical therapy. With trimethoprim-sulfamethoxazole and fluoroquinolones, the drug-bug mismatch was predicted to decrease 62% and 78%, respectively. Recurrent or persistent urinary tract infection and clinical sepsis were significantly correlated with specific clonotypes, especially with CH40-30. Furthermore, the examples demonstrate the ability to clonotype directly from patient urine samples within 1 to 3 hours of obtaining the specimen. In E. coli, subspecies-level identification by clonotyping can be used to significantly improve empirical predictions of antimicrobial susceptibility and clinical outcomes in a timely manner.

By predicting antimicrobial resistant isolates, CH clonotyping can limit treatment failures due to drug-bug mismatch and therefore reduce the further spread of resistant organisms. The data demonstrate that antimicrobial selections that resulted in a drug-bug mismatch were correlated with persistent or recurrent UTIs (as well as sepsis), as were broadly resistant clonotypes, such as CH40-30, and multidrug-resistant isolates more generally. By reducing the likelihood of a drug-bug mismatch, the clonotyping-guided approach to antimicrobial selection has a strong potential to reduce the likelihood of persistent or recurrent infection.

CH clonotyping provides a significant diagnostic advantage of high-resolution over traditional MLST. This is especially evident from the characterization of the CH40-30 clonotype from ST131. CH40-30. In this study, the inventors show for the first time that in addition to its resistance associations, this clonotype is heavily associated with persistent or recurrent UTIs and urosepsis. This is in sharp contrast to other clonotypes of ST131 that combined comprise 35% of the clonal group in the sample used here and that are not particularly distinct from the rest of the E. coli isolates with respect to either resistance or virulence pattern. Thus, such novel information about these CH clonotypes is very useful for studying the epidemiology and pathogenic mechanisms underlying UTIs, as well as for diagnostic and therapeutic purposes.

The relative simplicity and rapid nature of the qPCR and pyrosequencing protocols allow for an easy incorporation into the current clinical microbiology protocols and normal workflow. Depending on the proximity of the laboratory to the point of care (or clinical specimen collection), the clonotype identity can be defined within 1 to 6 hours of the patient providing the specimen. Organizing a proper feedback mechanism from the laboratory to the provider allows the physician to make more-accurate antibiotic selections. This could be done either for the initial prescription, within hours of the patient visit, or on the next day to correct the original prescription if a drug-bug mismatch is predicted (e.g., CH40-30 with fluoroquinolones, or CH35-27 with TMP-SMZ). Even in the latter scenario, clonotyping provides a 24- to 48-hour advantage over conventional antibiogram diagnostics, especially for those clonotypes with highly predictable resistance patterns. The currently estimated reagent cost of a single clonotype-specific qPCR test (less than or equal to $2) and of a broad-range clonotype-specific pyrosequencing run (less than or equal to $8) are sufficiently low to consider introduction of clonotyping into clinical diagnostics and to encourage its refinement sequencing,

Subspecies clonotyping to the level provided by or comparable to that of CH clonotyping provides many advantages to current diagnostics and might result in a paradigm shift in the management of infections caused by E. coli and other clonal bacterial pathogens. First, it provides prognostic power of antimicrobial resistance. Second, it allows for the genetic typing of clinical isolates to facilitate in-depth epidemiologic analyses of various clonotypes' associations with specific clinical outcomes in relation to treatment regimens, comorbidities, and patient demographics. Third, since clonotyping profiles are based on short DNA sequences, they are discrete and portable, making them suitable for analysis across laboratories, thereby conceivably allowing for the creation of global databases that could be queried by remote users. Subspecies clonotyping of bacterial pathogens provides a fast and cost-effective approach in routine clinical diagnostics, rapidly supplying practitioners with potentially critical information about the infecting strain.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

While the invention has been particularly shown and described with reference to an aspect and various alternate aspects, it will be understood by persons skilled in the relevant art that various changes in form and details can be made therein without departing from the spirit and scope of the invention. The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize

All references, issued patents and patent applications cited within the body of the instant specification are hereby incorporated by reference in their entirety, for all purposes. Aspects of the disclosure can be modified, if necessary, to employ the systems, functions, and concepts of the above references and application to provide yet further embodiments of the disclosure. These and other changes can be made to the disclosure in light of the detailed description.

### REFERENCES

Achtman M, et al. 1983. Six widespread bacterial clones among Escherichia coli K1 isolates. Infect. Immun. 39:315-335.
Barl T, et al. 2008. Genotyping DNA chip for the simultaneous assessment of antibiotic resistance and pathogenic potential of extraintestinal pathogenic Escherichia coli. Int. J. Antimicrob. Agents 32:272-277.
Carriço J A, et al. 2006. Illustration of a common framework for relating multiple typing methods by application to macrolide-resistant Streptococcus pyogenes. J. Clin. Microbiol. 44:2524-2532.
Caugant D A, et al. 1983. Genetic diversity and relationships among strains of Escherichia coli in the intestine and those causing urinary tract infections. Prog. Allergy 33:203-227.
Caugant D A, et al. 1985. Genetic diversity in relation to serotype in Escherichia coli. Infect. Immun. 49:407-413.
Clermont O, Bonacorsi S, Bingen E. 2000. Rapid and simple determination of the Escherichia coli phylogenetic group. Appl. Environ. Microbiol. 66:4555-4558.
Connell I, et al. 1996. Type 1 fimbrial expression enhances Escherichia coli virulence for the urinary tract. Proc. Natl. Acad. Sci. U.S.A. 93:9827-9832.
Dias R C, Moreira B M, Riley L W. 2010. Use of fimH single-nucleotide polymorphisms for strain typing of clinical isolates of Escherichia coli for epidemiologic investigation. J. Clin. Microbiol. 48:483-488.
Edelstein M, Pimkin M, Palagin I, Edelstein I, Stratchounski L. 2003. Prevalence and molecular epidemiology of CTX-M extended-spectrum beta-lactamase-producing Escherichia coli and Klebsiella pneumoniae in Russian hospitals. Antimicrob. Agents Chemother. 47:3724-3732.
Filatov D A. 2002. ProSeq: a software for preparation and evolutionary analysis of DNA sequence data sets. Mol. Ecol. Notes 2:621-624.
Hommais F, et al. 2003. The FimH A27V mutation is pathoadaptive for urovirulence in Escherichia coli B2 phylogenetic group isolates. Infect. Immun 71:3619-3622.
Hunter P R, Gaston M A. 1988. Numerical index of the discriminatory ability of typing systems: an application of Simpson's index of diversity. J. Clin. Microbiol. 26:2465-2466.
Johnson J R, Delavari P, Kuskowski M, Stepll A L. 2001. Phylogenetic distribution of extraintestinal virulence-associated traits in Escherichia coli. J. Infect. Dis. 183:78-88.
Johnson J R, Delavari P, O'Bryan T T. 2001. Escherichia coli O18:K1:H7 isolates from patients with acute cystitis and neonatal meningitis exhibit common phylogenetic origins and virulence factor profiles. J. Infect. Dis. 183:425-434.
Johnson J R, et al. 2008. Virulence genotypes and phylogenetic background of Escherichia coli serogroup O6 isolates from humans, dogs, and cats. J. Clin. Microbiol. 46:417-422.
Johnson J R, Owens K L, Clabots C R, Weissman S J, Cannon S B. 2006. Phylogenetic relationships among clonal groups of extraintestinal pathogenic Escherichia coli as assessed by multi-locus sequence analysis. Microbes Infect. 8:1702-1713.
Johnson J R, Russo T A. 2002. Extraintestinal pathogenic Escherichia coli: “the other bad E. coli.” J. Lab. Clin. Med. 139:155-162.
Johnson J R, Stepll A L. 2000. Extended virulence genotypes of Escherichia coli strains from patients with urosepsis in relation to phylogeny and host compromise. J. Infect. Dis. 181:261-272.
Maiden M C, et al. 1998. Multilocus sequence typing: a portable approach to the identification of clones within populations of pathogenic microorganisms. Proc. Natl. Acad. Sci. U.S.A. 95:3140-3145.
Manges A R, et al. 2001. Widespread distribution of urinary tract infections caused by a multidrug-resistant Escherichia coli clonal group. N. Engl. J. Med. 345:1007-1013.
Mendonça N, Leitao J, Manageiro V, Ferreira E, Canica M. 2007. Spread of extended-spectrum beta-lactamase CTX-M-producing Escherichia coli clinical isolates in community and nosocomial environments in Portugal. Antimicrob. Agents Chemother. 51:1946-1955.
Nicolas-Chanoine M H, et al. 2008. Intercontinental emergence of Escherichia coli clone O25:H4-ST131 producing CTX-M-15. J. Antimicrob. Chemother. 61:273-281.
Nowrouzian F L, Friman V, Adlerberth I, Wold AE. 2007. Reduced phase switch capacity and functional adhesin expression of type 1-fimbriated Escherichia coli from immunoglobulin A-deficient individuals. Infect. Immun. 75:932-940.
Ochman H, Selander R K. 1984. Standard reference strains of Escherichia coli from natural populations. J. Bacteriol. 157:690-693.
Ronald L S, et al. 2008. Adaptive mutations in the signal peptide of the type 1 fimbrial adhesin of uropathogenic Escherichia coli. Proc. Natl. Acad. Sci. U.S.A. 105:10937-10942.
Sokurenko E V, et al. 2004. Selection footprint in the FimH adhesin shows pathoadaptive niche differentiation in Escherichia coli. Mol. Biol. Evol. 21:1373-1383.
Stentebjerg-Olesen B, Chakraborty T, Klemm P 1999. Type 1 fimbriation and phase switching in a natural Escherichia coli fimB null strain, Nissle 1917. J. Bacteriol. 181:7470-7478.
Struelens M J. 1996. Consensus guidelines for appropriate use and evaluation of microbial epidemiologic typing systems. Clin. Microbiol. Infect. 2:2-11.
Suzuki S, et al. 2009. Change in the prevalence of extended-spectrumbeta-lactamase-producing Escherichia coli in Japan by clonal spread. J. Antimicrob. Chemother. 63:72-79.
Swofford D L. 2003. PAUP*: phylogenetic analysis using parsimony (* and other methods), version 4. Sinauer Associates, Sunderland, Mass.
Tamura K, Dudley J, Nei M, Kumar S. 2007. MEGA4: molecular evolutionary genetics analysis (MEGA) software version 4.0. Mol. Biol. Evol. 24:1596-1599.
Tartof S Y, Solberg O D, Riley L W. 2007. Genotypic analyses of uropathogenic Escherichia coli based on fimH single nucleotide polymorphisms (SNPs). J. Med. Microbiol. 56:1363-1369.
Tenover F C, et al. 1995. Interpreting chromosomal DNA restriction patterns produced by pulsed-field gel electrophoresis: criteria for bacterial strain typing. J. Clin. Microbiol. 33:2233-2239.
Touchon M, et al. 2009. Organised genome dynamics in the Escherichia coli species results in highly diverse adaptive paths. PLoS Genet. 5:e1000344.
Vejborg R M, Friis C, Hancock V, Schembri M A, Klemm P 2010. A virulent parent with probiotic progeny: comparative genomics of Escherichia coli strains CFT073, Nissle 1917 and ABU 83972. Mol. Genet. Genomics 283:469-484.
Weissman S J, et al. 2007. Differential stability and trade-off effects of pathoadaptive mutations in the Escherichia coli FimH adhesin. Infect. Immun 75:3548-3555.
Weissman S J, et al. 2006. Clonal analysis reveals high rate of structural mutations in fimbrial adhesins of extraintestinal pathogenic Escherichia coli. Mol. Microbiol. 59:975-988.
Wirth T, et al. 2006. Sex and virulence in Escherichia coli: an evolutionary perspective. Mol. Microbiol. 60:1136-1151.

1. A method of typing Escherichia coli in a sample comprising:
(a) determining a nucleic acid sequence in the sample of Escherichia coli (E. coli) type 1 fimbrial adhesion (fimH) gene and a further E. coli gene selected from the group consisting of fumC, adk, gyrB, icd, mdh, purA, and recA to identify a clonotype of the sample; and
(b) typing E. coli present in the sample based on the clonotype.

2. The method of claim 1, wherein the sample is a biological sample from a subject, and wherein the typing indicates presence of antibiotic resistant E. coli in the subject, or is used to diagnose or prognose a disease state in the subject.

3. The method of claim 2, wherein the typing indicates that the subject is infected with antibiotic resistant E. coli.

4. The method of claim 2, wherein the subject is at risk of having a urinary tract infection, and wherein the typing is used to diagnose a urinary tract infection in the subject.

5. The method of claim 2, wherein the subject is at risk of having sepsis, and wherein the typing is used to diagnose sepsis in the subject.

6. The method of claim 2, wherein the subject has a urinary tract infection, and wherein the typing is used to prognose the urinary tract infection in the subject.

7. The method of claim 2, wherein the subject has sepsis, and wherein the typing is used to prognose the sepsis in the subject.

8. The method of claim 3, wherein the typing indicates efficacy of an antibiotic treatment for the subject.

9. The method of claim 8, wherein the typing indicates efficacy of ampicillin (AMP), tetracycline (TET), ampicillin-sulbactam (A/S), trimethoprimsulfamethoxazole (T/S), amoxicillin-clavulanate (A/K), cefazolin (CZ), ciprofloxacin (CIP), gentamicin (GM), nitrofurantoin (NIT), ceftriaxone (CTR) and/or piperacillin-tazobactam (PTZ) in the subject.

10. The method of claim 2, wherein the typing is carried out after the subject has undergone treatment for the disease state or the infection with antibiotic resistant E. coli, and wherein the typing indicates efficacy of the treatment.

11. The method of claim 2, wherein the biological sample is selected from the group consisting of urine, blood, wound, tissue, saliva, sputum, feces, spinal fluid, plasma, peritoneal fluid, ascites, pleural fluid, joint fluid, abscess material, pus, tracheal secretions, bile, exudate, corneal scraping, bone, drainage and biopsy material.

12. The method of claim 2, wherein the biological sample is urine.

13. The method of claim 1, wherein the further E. coli gene is fumC.

14. The method of claim 1, wherein determining the fimH gene nucleic acid sequence comprises amplifying and determining a sequence of a portion of the fimH gene, wherein the portion of the fimH gene is amplified by an oligonucleotide primer pair consisting of 5′-CACTCAGGGAACCATTCAGGCA-3′ (SEQ ID NO: 01) and 5′-CTTATTGATAAACAAAAGTCAC-3′ (SEQ ID NO: 02).

15. The method of claim 14, wherein determining the fumC gene nucleic acid sequence comprises amplifying and determining a sequence of a portion of the fumC gene, wherein the portion of the fumC gene is amplified by an oligonucleotide primer pair that amplifies a fumC fragment of 500 nucleotides or less.

16. The method of claim 1, wherein determining the nucleic acid sequences comprises quantitative polymerase chain reaction (qPCR).

17. A composition consisting of between 2-5 oligonucleotide primer pairs, wherein:
(a) a first primer pair selectively amplifies a region of a fimH gene; and
(b) a second primer pair selectively amplifies a region of a gene selected from the group consisting of fumC, adk, gyrB, icd, mdh, purA, and recA.

18. The composition of claim 17, wherein the first primer pair consists of 5′-CACTCAGGGAACCATTCAGGCA-3′ (SEQ ID NO: 01) and 5′-CTTATTGATAAACAAAAGTCAC-3′ (SEQ ID NO: 02).
